(19) United States
(12) Patent Application Publication
(10) Pub. No.: US 2018/0298343 A1
(43) Pub. Date: Oct. 18, 2018

(54) COMPOSITIONS AND METHODS FOR A THREE DIMENSIONAL EX-VIVO GLOMERULAR CELL CO-CULTURE BIOLOGICAL ENGINEERING MODEL

(71) Applicant: Arunthathy Sivakumaran, Burlington (CA)

(72) Inventor: Arunthathy Sivakumaran, Burlington (CA)

(21) Appl. No.: 15/486,189

(22) Filed: Apr. 12, 2017

(51) Int. Cl. C12N 5/071; C12M 3/00; C12M 1/12; C12M 1/00; G01N 33/50; C12M 1/34

(52) U.S. Cl. CPC C12N 5/0686; C12N 5/0691; C12M 21/08; C12M 25/10; C12M 29/04; C12M 27/18; C12N 2513/00; C12M 41/46; C12M 23/20; G01N 33/5044; C12N 2502/28; C12N 2500/84; G01N 33/5082

(57) ABSTRACT

The invention provides compositions, methods and systems for the ex vivo co-culture of renal cells, more specifically, for the co-culture of glomerulus-derived vascular endothelial cells and podocyte cells using apparatus that mimics the in vivo cellular architecture of the renal corpuscle. The invention described herein finds a variety of uses, for example, as a model system for the study of renal corpuscle function, including the filtration of the blood supply that occurs at the interface of the glomerulus and Bowman's capsule, normal physiology of those cell types, and as a model system for the study of renal disease, including the study of drug effects on the functioning of these cell types.

### FIELD OF THE INVENTION

This invention provides apparatus, methods and systems for a three dimensional ex-vivo biological engineering model of a glomerulus. The ex vivo partitioned co-culture of glomerulus-derived vascular endothelial cells and podocyte cells, along with a glomerular basement membrane is used to create a model that mimics the in vivo three-dimensional cellular architecture of the glomerular filtration structure of the glomerulus-Bowman's capsule interface. This model enables the study of the effects of biological engineering variables i.e. shear rate (fluid mechanics), pressure, and mass transport e.g. of drugs, on glomerular model system function and cell physiology in health or disease conditions.

### BACKGROUND OF THE INVENTION

The kidney is one of a pair of organs in the back of the abdominal cavity, which has a number of significant roles. It acts as a filter, removing water soluble metabolic wastes (i.e. urea, ammonia) and foreign materials from the blood, which are excreted in the urine. It also has an endocrine function, which involves the production of hormones, including renin, erythropoietin and calcitrol and enzymes which are used for kidney feedback control, erythrocyte production and bone production, respectively. Additionally, the kidney regulates the volume of body fluids, as well as the osmolality, pH and electrolyte balance of the body fluids, and regulates blood pressure via control over the salt-water balance (Truskey, G. A. et al., 2009. “Transport Phenomena in Biological Systems,” Second Edition, publ. Pearson Education, Upper Saddle River, N.J.).

The component of the kidney where processing to form urine takes place is the nephron. Each of the approximately one million nephrons in each kidney consists of the renal corpuscle (containing the glomerulus and Bowman's capsule), the proximal convoluted tubule, the descending and ascending limbs of the loop of Henle, the distal convoluted tubule, and the collecting tubule (Truskey, G. A. et al., 2009. “Transport Phenomena in Biological Systems,” Second Edition, publ. Pearson Education, Upper Saddle River, N.J.).

FIG. 1 provides a schematic showing the general anatomy of the glomerulus-Bowman's capsule complex, i.e., the renal corpuscle 106, of the mammalian kidney. Collectively, the glomerulus 100 and surrounding Bowman's capsule 102 constitute a renal corpuscle 106. The renal corpuscle 106 consists of the capillary-rich glomerulus structure 100, and the Bowman's capsule 102 which encases and is in close physical association with the glomerulus 100. Blood, indicated by a stippled arrow, is brought to the glomerulus 100 via the afferent arteriole 108, where it branches into the glomerular capillary 104 network and leaves via the efferent arteriole.

The role of the glomerulus is to function as a filter. As the blood flows through the glomerulus capillaries 104 under a positive pressure, about one-fifth of the blood is filtered through the permeable walls of the glomerular capillaries, shown by the filled arrows, to form a glomerular filtrate, which passes into the Bowman's capsule space 120. After the blood passes through the glomerulus 100, the fraction that remains after this initial filtration exits the kidney, indicated by a cross-hatched arrow, via an efferent arteriole 110.

Cellular components of the blood remain in the glomerular capillary blood flow, along with large proteins such as albumin. From the Bowman's capsule 102, the glomerular filtrate, indicated by the empty arrows, empties into the proximal tubule of the nephron duct system for further processing. After passage through the proximal tubule, the filtrate enters the loop of Henle, the distal tubule, and finally into the collecting tubule leading to the bladder. As the glomerular filtrate passes through the nephron after leaving the renal corpuscle, most of the water and some of the solutes from the filtrate are reabsorbed into the blood supply via the peritubular capillaries. The remaining water and concentrated solutes in the nephron tubules become urine (Truskey, G. A. et al., 2009. “Transport Phenomena in Biological Systems,” Second Edition, publ. Pearson Education, Upper Saddle River, N.J.).

The interface between the glomerular capillaries 104 and the Bowman's capsule 102 is a complex structure that regulates the formation of glomerular filtrate. A portion of this interface 130 in FIG. 1 is depicted schematically in FIG. 10. Critical components in this structure include at least two cell types, and a glomerular basement membrane or GBM. The glomerular endothelial cell layer 156 is attached to the GBM 121 on the side facing the glomerular capillary lumen, and the podocyte cells 160, which are highly specialized visceral epithelial cells, are attached to the other side of the GBM 121 and face the Bowman's capsule space 152 (Truskey et al., 2009. “Transport Phenomena in Biological Systems,” Second Edition, publ. Pearson Education, Upper Saddle River, N.J.).

The glomerular basement membrane is an extracellular matrix that is characterized by specific proteins, including nidogens, heparin sulfate proteoglycans, laminins and collagen type-4. The adult laminins are deposited by the combined action of the endothelial cells 156 and podocyte cells 160, whereas the adult collagen type-4 proteins are deposited by the podocytes alone (Miner, J. 2012. “The glomerular basement membrane,” Exp Cell Res, 318 (9): 973-978.

The combined structure of the glomerular vascular endothelial cells, the podocyte cells and the glomerular basement membrane form the filtration apparatus that regulates the composition and flow of glomerular filtrate into the downstream nephron tubule system, initially into the proximal tubule portion of the nephron, shown by the empty arrow in FIG. 1. This glomerular filtration apparatus allows the passage of low molecular weight substances, e.g., amino acids, peptides, glucose, ions and metabolic waste products, from the glomerular blood supply (see 150 in FIG. 10) and into the Bowman's capsule space (see 152 in FIG. 10). This same filtering mechanism excludes cellular blood components such as erythrocytes and white blood cells and large proteins, e.g., albumin and immunoglobulins.

A simplified view of the glomerular filtration structure comprises three layers, which are (i) the glomerular endothelial cells, (ii) the glomerular basement membrane, and (iii) the podocytes. Recent studies reveal that the structure is more complex, additionally incorporating an endothelial surface layer (ESL) 154 and a subpodocyte space (SPS) (Sekulic, S. and M. Sekulic. 2015. “Rheological influence upon the glomerular podocyte and resultant mechanotransduction,” Kidney Blood Press Res, 40: 176-187.)

The ESL 154 is an approximately 500 nm layer composed of a hydrated web-like structure on the luminal side of the glomerular endothelium that consists of two components. The first is the glycocalyx, consisting of negatively charged glycoproteins, which are connected by proteoglycans and glycoproteins to the glomerular endothelial luminal surface. The glycocalyx equally covers both the fenestrae (pores that exist between the endothelial cells) and the interfenestral domains. The glycocalyx is attached to the second component, the cell coat. The latter consists of adsorbed plasma proteins, secreted proteoglycans, glycosaminoglycans and glycoproteins. The removal of the majority of the glycocalyx has been found to significantly increase passage across the barrier of small molecules, water and albumin (Singh, A. et al., 2007. “Glomerular endothelial glycocalyx constitutes a barrier to protein permeability,” J Am Soc Nephrol, 18: 2885-2893).

The SPS is formed when processes from the cell body of the podocyte are anchored to the GBM, enclosing in some cases a space underneath the cell body and above the GBM called the SPS. This space represents 60% of the filtration surface. It was found that this pathway through the subpodocyte space and through the exit pores causes resistance of flow and a different solute permeability than the flow that passes through the slit diaphragm straight through the Bowman's space (Sekulic, S. and M. Sekulic. 2015. “Rheological influence upon the glomerular podocyte and resultant mechanotransduction,” Kidney Blood Press Res, 40: 176-187).

The rate of formation of glomerular filtrate is historically a measure of kidney health and disease. The glomerular filtration rate (GFR) represents the collective volumetric flow rate of all the kidneys' glomeruli, estimated to be between 120-125 mL/min (Guyton, A. C and J. E. Hall. 2013. “Textbook of medical physiology—11^{th} ed.,” Elsevier-Saunders, New York). This rate is maintained at a relatively constant value by various regulatory mechanisms.

Traditional adherent cell culture, e.g., as might be used to propagate glomerulus-derived vascular endothelial cells or podocyte cells, uses a two-dimensional surface onto which the cells adhere, and are nourished by an overlying culture media in a static environment. These types of culture systems are problematic because they are greatly limited in their ability to mimic more dynamic in vivo three-dimensional cell environments. As a consequence, adherent cells that are grown in static culture are much less likely to display the normal physiology and cell functions that exist in vivo.

In contrast, various types of perfusion bioreactors can be advantageously used to culture cells in dynamic three dimensional environments. See Sanyal, S. 2014. “Culture and assay systems used for 3D cell culture,” review article, Corning Life Sciences application note 245, document no. CLS-AC-AN-245 (publ. Corning Life Sciences). As used herein, the expressions “perfusion,” perfusion culture,” “perfusion system” or “perfusion apparatus” refer to any type of cell culture apparatus that continuously delivers culture media to cells, where the culture media constantly moves over or through cultured cells, and continuously eliminates spent media. This enables the cells to be kept in culture without the need to re-inoculate or otherwise seed a new cell culture. There is also the advantage that the lag phase is eliminated, the cell densities achievable are greater than non-perfusion systems and the cells can be cultured over much longer periods of time (up to months), owing to the fact that metabolic wastes do not accumulate and the cell culture media nutrients are kept close to optimal levels (Langer, E. S. and R. A. Rader. 2014. Bioprocess Journal, Spring: 50-55; Sargent, B. 2013. “Perfusion bioreactors: with so much to offer they deserve a closer look.” Cell Culture Dish.)

Various perfusion culture techniques are known. For example, hollow fiber bioreactor designs (termed HFBR) using internal capillary fibers or membranes to culture cells to high densities in continuous cell cultures are frequently used. These hollow fiber bioreactors use synthetic fibers, also termed capillaries, made from porous synthetic materials that pass through a sealed chamber or cartridge. Generally, cells of interest are seeded and cultured on the outside of the hollow fibers in the extra-capillary space (ECS) within the sealed cartridge. Cell culture medium containing necessary nutritional components is pumped into one end of the sealed system into the luminal spaces of the fibers, travels along the length of the fiber lumen, and is collected at the terminus of the fibers, and is then channeled out of the cartridge. During this transit, a fraction of the culture medium traverses the porous, semi-permeable fiber walls, thereby providing nutrition not only to the layer of cells that comes in direct contact with the fiber wall, but also to cells which reside in deeper cell layers (Cadwell, J. 2004. “New developments in hollow fiber cell culture.” American Biotechnology Laboratory).

HFBR perfusion systems have the advantage of continuously providing nutrition to the cells grown on the outside of the hollow fibers by diffusion of culture medium components from the fiber lumen into the extra-capillary space where the cells are cultured. There is also a benefit of removing metabolic waste products such as lactic acid from the cell culture that diffuses in the opposite direction, i.e., from the extra-capillary space into the medium flow stream in the fiber lumen (Cadwell, J. 2004. “New developments in hollow fiber cell culture.” American Biotechnology Laboratory).

HFBR perfusion bioreactors can be operated as batch mode systems, i.e., recirculated (i.e., full retentate recycle) culture systems, where the culture medium that flows through the hollow fiber is recycled back into a central reservoir that resupplies the cartridge feed line.

Alternatively, the HFBR perfusion bioreactors can be operated as continuous systems, where the expended culture medium that flows through the hollow fibers is not recycled back into a feed reservoir, and the system is continuously replenished with fresh culture medium.

Various attempts have been made to model structural and functional aspects of the mammalian kidney in the hope of creating improved techniques for clinical kidney dialysis or for the purpose of creating model systems that are amendable to the study of renal physiology and renal disease.

U.S. Pat. No. 3,483,867 to Markovitz (Markovitz, M. “Artificial glomerulus and a method for treating blood. U.S. Pat. No. 3,483,867, Jun. 13, 1968) describes a cell-free artificial glomerulus filtration apparatus for the filtration of whole blood for use by patients requiring traditional kidney dialysis.
Fournier (1999), “Basic Transport Phenomena in Biomedical Engineering,” Taylor & Francis, Philadelphia, Pa., 1999, 312 pages total, see pgs. 282-284, published in Journal of Controlled Release 67(s 2-3):417 (July 2000) describes a cell-free, naked hollow fiber membrane to purify blood to approximate a functional endpoint of kidney processing in the hope of developing an artificial organ.
Lee and Choi (2012), “The artificial glomerulus design using diffusion in microchannels,” International Journal of Precision Engineering and Manufacturing, 13(2):307-310 describes a cell-free “artificial glomerulus” device for the separation of blood plasma components by a diffusion mechanism through microfluidic channels in the absence of any type of membrane and without use of a dialysate.

Various references describe the co-culture of endothelial cells and podocyte cells using static cell culture systems. See, for example, Rastaldi, M and M. Li. “Method for the three-dimensional co-culture of podocytes and endothelial cells and relative in vitro co-culture system”. Patent Application Publication, Pub no.: US 2013.0177929 A1, Jul. 11, 2013; Li et al., 2016. “Three-dimensional podocyte-endothelial cell co-cultures: Assembly, validation, and application to drug testing and intercellular signaling studies,” Eur. J. Pharm. Sci., 86:1-12; Slater et al. (2011), “An In Vitro Model of the Glomerular Capillary Wall Using Electrospun Collagen Nanofibres in a Bioartificial Composite Basement Membrane,” PLoS ONE 6(6): e20802;

Bruggeman et al., 2012. “A cell culture system for the structure and hydrogel properties of basement membranes; Application to capillary walls,” Cell. Mol. Bioeng., 5(2): 194-204 describes a system for the co-culture of podocyte cells and endothelial cells using a hydrogel scaffold without a rigid support. Bruggeman et al., (2012) tests the hydrogel membrane scaffolds in a perfusion chamber under net positive pressure to simulate glomerular filtrate production. This is a model with non-physiological flow, where the directional flow is ‘normal’, or 90° to the filter.

Various publications describe the culture of renal proximal tubule cells (rather than glomerular cells) on various apparatus and substrates with the goal of making a device or culture system for the study of kidney proximal tubule cell physiology or improved kidney dialysis techniques. See, for example, Zink and Zay. “Bioreactor unit for use in bioartificial kidney device,” US Patent Appl. Publication No. US2015/0076066; Zinc and Li, “In vitro assay for predicting renal proximal tubule cell toxicity,” US Patent Appl. Publication No. US2015/0197802; and Tasnim et al. (2010), “Achievements and challenges in bioartificial kidney development,” Fibrogenesis & Tissue Repair, 3:14

Various publications generally describe hollow fiber bioreactors adapted for cell co-cultures employing endothelial cells. See, for example: Redmond, E. and P. Cahill. 1995. “Perfused transcapillary smooth muscle and endothelial cell co-culture—a novel in vitro model,” In Vitro Cell. Dev. Biol. Anim., 31: 601-609; Redmond et al., 1998. “Flow-mediated regulation of G-protein expression in co-cultured vascular smooth muscle and endothelial cells,” Arterioscler. Thromb. Vasc. Biol., 18: 75-83; Cadwell, J. 2012. “The hollow fiber bioreactor and cell co-cultivation,” American Laboratory article; FiberCell Systems, Inc., 2014. “Endothelial Cartridge Instructions,” Revision 6.0, dated Jan. 8, 2014.

What is needed in the art are improved cell culture model systems for studying the effects of biological engineering critical variables on glomerular system function, i.e. the filtration mechanism that generates the glomerular filtrate in vivo. What is needed in the art are improved apparatus and model systems for the perfusion-style (i.e., non-static) partitioned co-culture of glomerulus-derived vascular endothelial cells and podocyte cells. What is needed in the art is a method of studying the effects of cell culture parameters or biological engineering critical variables on the cell physiology of these cell types. What is needed in the art is a 3-dimensional model system with physiologically relevant tangential flow directed parallel to the membrane filter. What is needed are improved model systems for understanding the three-dimensional architecture at the interface of the glomerulus and Bowman's capsule, more specifically, involving the endothelial cells that reside in the glomerulus vascular network, podocyte cells, and further incorporating the glomerular basement membrane that exists between those adjacent cell types. What is needed in the art are improved model systems for the understanding of the effects of drug toxicity on the endothelial cell and podocyte cell physiology and the glomerular system function. What is needed in the art are improved model systems for understanding renal (glomerular) disease that arises from malfunction in the glomerular filtration process, and model systems for studying endothelial cell and podocyte cell physiology and glomerular system function in response to treatments, such as drug treatments.

The present invention, in its many embodiments, provides compositions and methods that overcome these challenges in the art, have a number of advantages over the state of the art and provide many benefits previously unrealized in products and methods currently used in the art. Still further benefits flow from the invention described herein, as will be apparent upon reading the present disclosure.

### SUMMARY OF THE INVENTION

In its broadest aspect, the invention provides apparatus, methods and systems for a three dimensional ex-vivo biological engineering model that mimics glomerular structure and function. The invention provides ex vivo partitioned co-cultures of glomerulus-derived vascular endothelial cells and podocyte cells, along with the glomerular basement membrane, that are used to create a model that mimics the in vivo three-dimensional cellular architecture of the glomerular filtration structures of the glomerulus-Bowman's capsule interface.

The invention finds a variety of uses and benefits, for example, as a model system for (i) the study of normal and diseased cell physiologies of vascular endothelial cells and podocyte cells, (ii) as a three dimensional biological engineering model system for ex vivo replication of the glomerular filtration function, (iii) for the simulation and study of the effects of a number of biological engineering considerations, e.g., shear rate, pressure, fluid rheology and mass transport, i.e., of drugs on glomerular filtration function and cell physiology (iv) as a model system for the study of kidney (glomerular) disease effects on glomerular function and cell physiology, (v) to study the effects of drugs that are intended to treat kidney disease on glomerular function and cell physiology, (vi) for testing of any types of drugs to predict their potential to induce renal toxicity and negatively impact the glomerular filtration function if administered to a patient, and (vii) for screening test compounds in an effort to identify therapeutic agents that have the ability to improve renal (glomerular) function in disease conditions. One of skill in the art will recognize the wide applicability and benefits of the invention, not limited to the aforementioned.

The invention also provides means for conducting pharmacodynamic studies to determine the relationship between the drug concentration and its effect on the glomerulus model. The effects can be measures of function, such as the permeate flow rate (ml permeate/min, analogous to the in vivo glomerular filtration rate, or GFR), albumin sieving coefficient/albumin permeate concentration or measures of cell physiological state, i.e. any suitable cell physiological marker.

In one aspect, the invention provides dynamic (i.e., non-static) partitioned cell co-cultures that use an apparatus having an enclosed housing, termed a cartridge, and at least one and up to many thousands, typically less than 50 semi-permeable hollow fibers disposed within the cartridge (i.e., the fiber is within the cartridge housing). The fiber has an exterior surface and an interior surface, and an interior lumen (also termed a luminal space, or simply lumen) bounded by the interior surface of the hollow fiber, and is further characterized where the termini of the fibers are fused to each other in a manner such that when the medium is delivered into a feed port in the cartridge, the medium flows through the luminal spaces of each of the fibers, and that flow is converged again to exit the cartridge at an exit port.

In this system, glomerulus-derived vascular endothelial cells are seeded and cultured within the fiber lumen on the inner wall of the hollow fibers, and podocyte cells are cultured within the extra-capillary space (ECS) on the outside surfaces of the hollow fibers. The cells are associated with the fiber walls, and can be cultured associated with the fiber walls either in the absence or presence of precoated ECM. The cell culture medium that is delivered to the cartridge is not directly sent to the extra-capillary space. The semi-permeable walls of the hollow fibers permit the passage of water and dissolved solutes below a specified molecular weight threshold from the fiber lumen and into the ECS, and prevent the traversal of larger molecular weight proteins and whole cells.

The portion of the culture medium, for example when the culture medium is whole blood, that has traversed the full length of the fiber luminal spaces, that is to say, medium that entered the feed port (also termed the inlet port) of the bioreactor cartridge, passes through the full length of the hollow fiber luminal spaces and exits by the retentate port (also termed the outlet port or exit port) is termed the retentate or retentate flow.

The portion of the culture medium that crosses the semi-permeable walls of the hollow fibers and passes into the ECS is termed the permeate, and can be diverted as a permeate flow from the ECS through a permeate port that passes through the outer wall of the bioreactor cartridge and accesses the ECS.

The whole blood that has traversed the full length of the fiber luminal spaces, that is to say, medium that entered the feed port (also termed the inlet port) of the bioreactor cartridge and exited the retentate port (also termed the outlet port or exit port) can be diverted and collected for any type of sampling or testing. This material that passes through the full length of the hollow fiber luminal spaces is termed the retentate. The retentate can be captured in a retentate reservoir, which is in fluid communication with and downstream of the hollow fiber interior luminal spaces, and/or alternatively, recycled for reentry back into the reactor cartridge through the central/feed reservoir and the feed port. Similarly, the permeate can be optionally be captured in a permeate reservoir, and/or alternatively, recycled for reentry back into the reactor cartridge through the feed port. A permeate reservoir is in fluid communication with and downstream of the extra capillary space.

The cartridge is typically supplied by a feed line connected to a medium reservoir, i.e., the cartridge and the culture medium reservoir are in fluid communication with each other. There is a directional flow of the cell culture medium through the fiber lumens, propelled by any suitable pump mechanism, such as a peristaltic pump or a pulsatile pump. The culture systems of the invention can further contain a diafiltration reservoir connected to the culture medium reservoir to supply the culture medium reservoir with various components to be added to the culture medium.

The porosity of the fiber walls can be specified to any desired size. For example, a semi-permeable wall of the hollow fiber can be selected such that the culture retentate retains at least 90% of albumin protein contained in the whole blood culture medium. In other embodiments, a semi-permeable wall of the hollow fiber can be selected having any desired molecular weight cut-off (MWCO) value, for example, a MWCO value of 5 kilodaltons with 90% retention efficiency of any component of the culture medium that is larger than 5 kilodaltons.

In other embodiments, the fiber walls can optionally be coated with any suitable natural, i.e., naturally occurring, or artificial materials that serves as an extracellular matrix material (ECM) to aid in cell attachment or cell growth, and/or where the ECM can structurally contribute to the selectivity or rate of the perfusion filtering function that occurs when components of the culture medium pass from the luminal spaces into the extra-capillary space to form a permeate. In some embodiments, the use of extracellular matrix (ECM) material on the fiber walls mimics the in vivo glomerular basement membrane (GBM). In various embodiments, the ECM coating is optionally applied to either the exterior surface of the hollow fiber, the interior surface of the hollow fiber, or to both surfaces. When both the exterior surface and the interior surface of the hollow fiber are treated with an ECM material, the same ECM material need not be used on both of these surfaces.

The cell culture medium that is used in the co-culture system is not particularly limited. The culture medium can optionally be a whole blood, a chemically defined cell culture medium, either with or without supplementation with a blood product, such as blood serum or blood plasma, or alternatively, supplemented with a suitable serum substitute, as known in the art, for example, supplemented with a platelet lysate preparation or with an artificial, chemically defined serum replacement. In other embodiments, cells are cultured in a defined culture medium that has been supplemented with whole blood. In some embodiments, the cell culture medium is a mesangial cell-conditioned cell culture medium, or is fresh medium that has been supplemented with a fractional portion of mesangial cell-conditioned culture medium. In some embodiments, the cell culture medium comprises albumin protein, and where the semi-permeable walls of the hollow fibers retain at least 90% of albumin protein contained in the culture medium.

The co-culture bioreactors of the invention can be operated as batch mode systems, i.e., the retentate is recycled and recirculated, where the culture medium that flows through the hollow fiber is recycled back into a central reservoir that resupplies the cartridge feed line. One possible batch-mode configuration is shown in FIG. 5. Alternatively, the co-culture bioreactors of the invention can be operated as continuous systems, where the expended culture medium that flows through the hollow fibers is not recycled back into a feed reservoir, and the system is continuously replenished with fresh culture medium. Various continuous systems are shown in FIGS. 7 and 9.

The cell co-cultures of the invention are typically established using a suitable cell culture medium in a batch mode, with no collected permeate, as shown in FIG. 5. Following establishment, the glomerular cell co-culture model system is operated with permeate flow in one of the configurations shown in FIGS. 6, 7, 8 and 9. Similarly, following establishment, the type and/or formulation of the cell culture medium can optionally be changed, for example, to utilize a whole blood culture system.

Cell cultures comprising any suitable apparatus as described herein and inoculated with the co-cultured cell types described herein are a feature of the invention. Furthermore, where the apparatus has been modified, enhanced or optimized for the co-culture of the cell types described herein, such apparatus is also a feature of the invention. Apparatus alone, for example any of the apparatus described herein, in the absence of the cultured cells, is also a feature of the invention.

The vascular endothelial cells and podocyte cells that find use with the invention are not particularly limited, and can be, for example, primary cells or finite cell cultures. In other embodiments, continuous cell lines having the capacity for indefinite subculture are used; such cell lines are frequently termed established cell lines or immortalized cell lines. Transformed cell lines also find use with the invention. Cells from a variety of sources find use with the invention, including cells derived from humans, cells derived from diseased humans, cells derived from transgenic animals, cells derived from healthy animals, cells derived from diseased animals, and cells carrying one or more genetic anomaly (e.g., a naturally arising mutation or an engineered mutation such as found in a knock-out model animal).

In other aspects, the invention provides methods for studying the process of glomerular filtrate production that occurs at the glomerulus-Bowman's capsule interface. The invention in its various forms provides a model system that mimics the in vivo glomerular filtration function. The co-culture system of the invention has key architectural similarities to the naturally occurring filtration apparatus in the glomerulus-Bowman's capsule complex. Methods of the invention for the study of healthy cells and disease conditions utilize the cultures and apparatus described in the present disclosure. These model systems of the invention also find use in studying the effects of known drugs on glomerular filtration function and the cell types described herein, as well as on the glomerular basement membrane (GBM). Furthermore, they can also be adapted for use in methods to identify new drugs that regulate glomerular filtrate production and can be used to treat human kidney disease, i.e., the invention provides apparatus and methodology for drug screening to identify candidate drugs for the treatment of glomerular based kidney disease.

In some aspects, the invention provides methods for assessing a functional response or cell physiological response following a treatment of the partitioned co-culture system described herein. The functional or cell physiological response that is assessed can be any type of functional measure or cell physiological effect in response to any kind of treatment, stimulus or culture condition. The physiological effect can include any change in a molecular marker, a measurable or observable physiological trait, or some other physiological effect or physiological property. A variety of physiological markers or traits can be used to monitor the effects of treating the cell co-culture system. In some embodiments, that marker is a molecular marker, and can be a protein marker or a nucleic acid marker. Molecule markers can be selected that are indicative, for example, of cell health, cell differentiation, cell disease or cell toxicity. The marker can be assessed by analysis of the whole blood retentate, permeate, or by analysis of cellular material, depending on the marker. In other aspects, a system functional measure is a characteristic feature of the glomerular co-culture model system, such as but not limited to, the rate of permeate flow, a podocyte cell count (i.e. in the permeate), permeate marker concentration, or a sieving coefficient of a marker in the retentate and permeate fractions, such as albumin or total protein. A cell physiological marker or functional measure is first assessed (e.g., measured) prior to initiating a treatment in the co-culture system, and is measured again, optionally during treatment, and after the treatment of the co-culture system. By comparing the cell physiological marker or functional measure value prior to initiating the treatment to the physiological marker or functional measure value at a time after initiating the treatment in the glomerular cell co-culture model system, a physiological or system functional response is determined. In some aspects, the treatment is a drug treatment. Other types of treatments are the altering of a cell culture parameter or the altering of a biological engineering variable (via changing an equipment parameter), such as modifications of the pressure of the whole blood passing through the hollow fiber luminal space.

### BRIEF DESCRIPTION OF THE DRAWINGS

FIG. 1 provides a schematic showing the general anatomy of the glomerulus and Bowman's capsule complex of the mammalian kidney, which consists of the glomerulus capillary tuft and Bowman's capsule.

FIG. 2 provides an illustration of a hollow fiber bioreactor cartridge unit, where the cartridge forms a part of the partitioned cell co-culture system of the invention. The particular cartridge illustrated is populated with glomerulus-derived vascular endothelial cells 216 within the hollow fiber luminal space 212 and podocyte cells 218 within the extra-capillary space 214.

FIG. 3 provides a schematic of a hypothetical lateral transect of the hollow fiber bioreactor cartridge unit shown in FIG. 2, along the dashed transect line 230 shown in FIG. 2 where this cartridge forms a part of the partitioned cell co-culture system of the invention. The schematic of FIG. 3 is not drawn to scale. For illustration purposes, only three hollow fibers are shown in the transect view.

FIG. 4 provides a schematic of a hypothetical longitudinal transect of a single hollow fiber contained in the bioreactor cartridge unit shown in FIG. 2. This single hollow fiber forms a part of the partitioned cell co-culture system of the invention. For illustration purposes, only one hollow fiber is shown in the longitudinal transect view of FIG. 4. The schematic of FIG. 4 is not drawn to scale. Coatings of an artificial or natural extracellular matrix material 430 are shown. Basement membrane material 422 and 424 optionally deposited by vascular endothelial cells and the podocyte cells, respectively, is also indicated, although it can displace the coatings shown in 430.

FIG. 5 provides a generalized schematic of a hollow fiber bioreactor that forms part of the partitioned cell co-culture system of the invention. This is the configuration used to allow the cell co-culture to be established using the cell culture medium. The hollow fiber bioreactor illustrated in the figure is a batch (or 100% retentate recycled) bioreactor system, where the retained portion of the cell culture medium 562 that passes through the full luminal length of the hollow fibers, passes through exit port of the bioreactor cartridge and is recirculated to feed the hollow fiber cartridge again via the feed reservoir 540. There is no permeate stream in this configuration.

FIG. 6 provides a schematic of a variation of the partitioned cell co-culture system of the invention as described in FIG. 5. Here, there is permeate flow and hence, this schematic represents a system that can be used to model the glomerular co-culture system. This cell culture apparatus further contains a permeate collection reservoir that captures the component from the whole blood that passes from the fiber luminal space, through the semi-permeable walls of the hollow fibers and into the extra-capillary space. The flow rate of a whole blood fraction (with proteins and molecules having a MWCO below that of the hollow fiber membrane) that is pumped from the diafiltration reservoir is matched to the permeate flow rate, preventing the concentration of the cells and blood proteins in the retentate above physiological conditions (i.e. above the molecular weight cut off (MWCO) size of the hollow fiber membrane).

FIG. 7 provides a schematic of a variation of the partitioned cell co-culture glomerular model system of the invention (continuous flow mode), where the cell culture apparatus contains a collection reservoir that collects and isolates the whole blood fraction termed the permeate that passes through the hollow fiber luminal space and into the extra-capillary space 714. The retentate reservoir 780 captures the whole blood fraction that traverses the length of the hollow fiber luminal space from the cartridge feed port to the cartridge retentate collection port.

FIG. 8 provides a schematic of a variant of the partitioned glomerular cell co-culture model system, where the system operates in a batch mode where both the permeate stream 873, 883 and the retentate stream 803 are 100% recycled into a feed reservoir that supplies the feed line to the bioreactor cartridge.

FIG. 9 provides a schematic of a variant of the partitioned glomerular cell co-culture model system shown in FIG. 7, where the system has been adapted to accommodate testing the effects of drugs. This system contains multiple reservoirs for whole blood alone 960 and whole blood with particular drugs 950, 990. These reservoirs are in fluid communication with the feed reservoir 940, allowing different combinations of drugs to be pumped into the feed reservoir 940 by regular or programmable pumps 954, 964, 994. A programmable pump 944 can also allow different drug dosing intervals and profiles to be introduced into the glomerular cell co-culture model system.

FIG. 10 provides a schematic showing the general anatomy of the glomerulus and Bowman's capsule interface of the mammalian kidney. This view is an expanded view of the portion of this interface indicated by the dashed rectangle 130 shown in FIG. 1.

### DETAILED DESCRIPTION OF THE INVENTION

Most broadly, the invention provides techniques and apparatus for a biological engineering model that involves the ex vivo partitioned co-culture of two principle cell types that lie at the interface of the glomerulus and Bowman's capsule in the mammalian kidney. This is the in vivo site of blood filtration that produces a glomerular filtrate in the generation of urine. The two cell types used in the model are the glomerulus-derived vascular endothelial cells and the podocyte cells, with a glomerular basement membrane between the cell types. The invention provides compositions, methods and systems in order to create a biological engineering three-dimensional ex vivo co-culture model of these cell types that mimics the three-dimensional architecture of the glomerular filtration system.

The invention provides a three-dimensional ex-vivo model system for the study of the effects of a number of biological engineering variables. These include but are not limited to: shear rate (i.e. fluid mechanics), pressure (e.g. blood pressure inside the hollow fibers, mimicking pressure inside the glomerular capillaries or e.g. the net filtration pressure), rheological character of the fluid (i.e. fluid dynamics) and mass transfer of e.g. drugs (mass transfer-transport phenomenon) on the glomerular filtration function. The model systems of the invention are adaptable to permit the modeling of normal glomerular function as well as aberrant renal function as occurs in glomerular disease. The model systems of the invention enable the investigation of the effects of disease and drugs on the system function, as well as the cellular and molecular physiology of the endothelial and podocyte cells.

The model systems of the invention incorporate a three to five-layer glomerular filter structure. The invention encompasses specialized apparatus for the co-culture of glomerular endothelial and podocyte cell types, the actual cell cultures which include the apparatus and the cells populating the apparatus, and methodologies that incorporate the populated culture apparatus. The invention also includes an artificial or natural glomerular basement membrane (GBM), including the natural secretion of this layer by the cell types cultured. The invention also encompasses the use of cell culture medium or whole blood or blood product that has been supplemented with various growth factors, such as those that are secreted by glomerular mesangial cells, or alternatively, cell culture medium or whole blood or blood product that has been conditioned by exposure to a mesangial cell culture, and as a result, contains mesangial cell secreted factors.

### GENERALLY

The co-culture systems of the invention are asymmetric co-cultures of two cell types that are segregated in distinct partitions of the culture apparatus, but remain in diffusible communication. The co-culture systems of the invention are dynamic model systems that keep culture medium or whole blood traversing in tangential flow through the culture apparatus.

The co-culture systems of the invention involved in the cell culture establishment (FIG. 5) is a tangential flow system that incorporates a flow of culture medium through hollow fiber capillaries within a bioreactor cartridge, where that flow of culture medium feeds the cells growing on the interior (i.e., luminal) surface of the capillaries, as well as perfuses through the porous walls of the hollow fiber capillaries and into the extra-capillary space to provide nutrition to the cells growing on the outer surface of the capillaries. Furthermore, old culture medium previously exposed to the growing cells, which now contains cellular waste products and is deficient in nutrient content, is removed (in part or in whole) and new culture medium delivered to the growing cells.

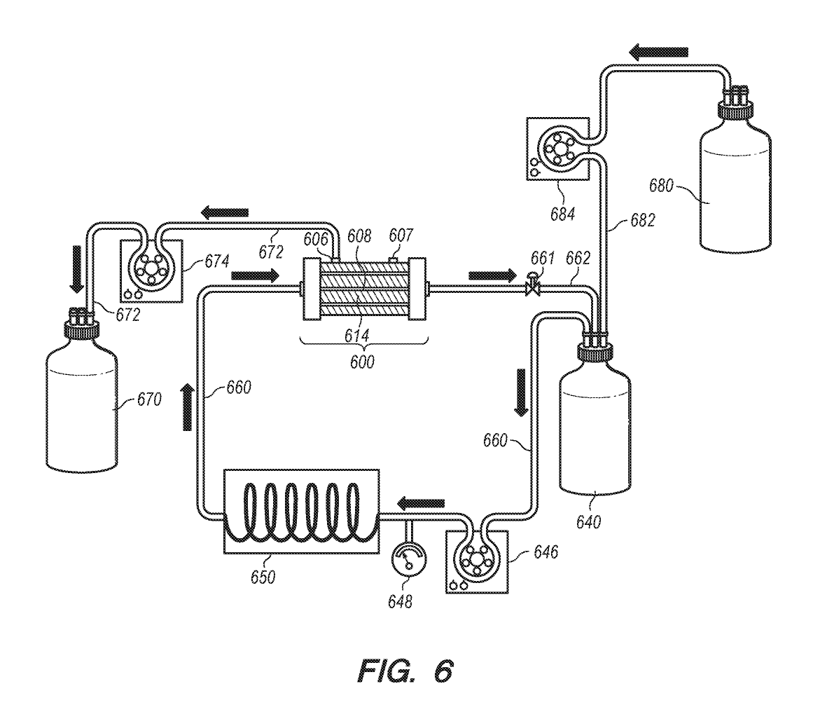

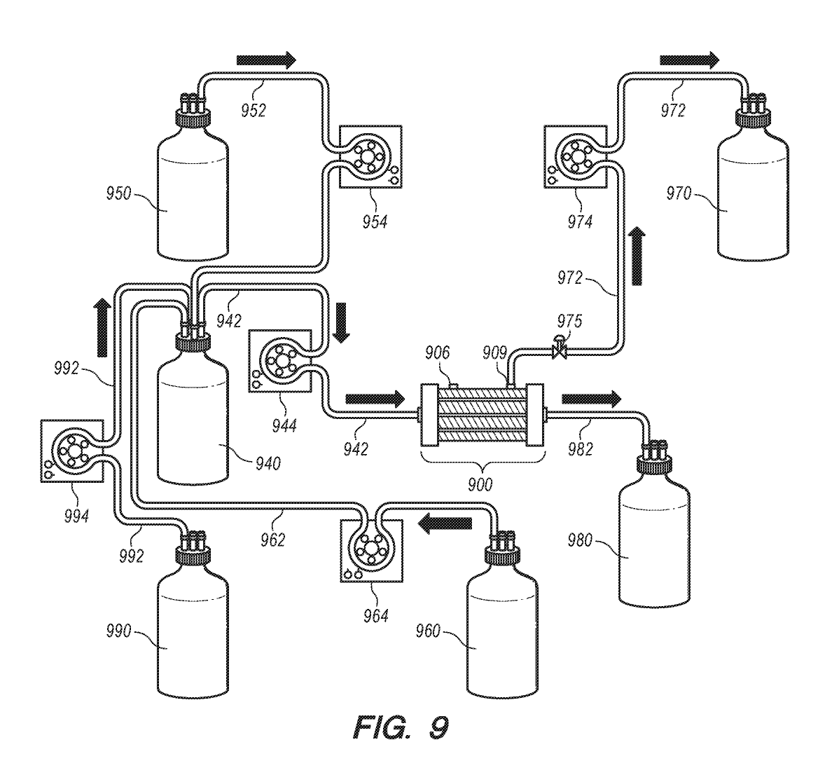

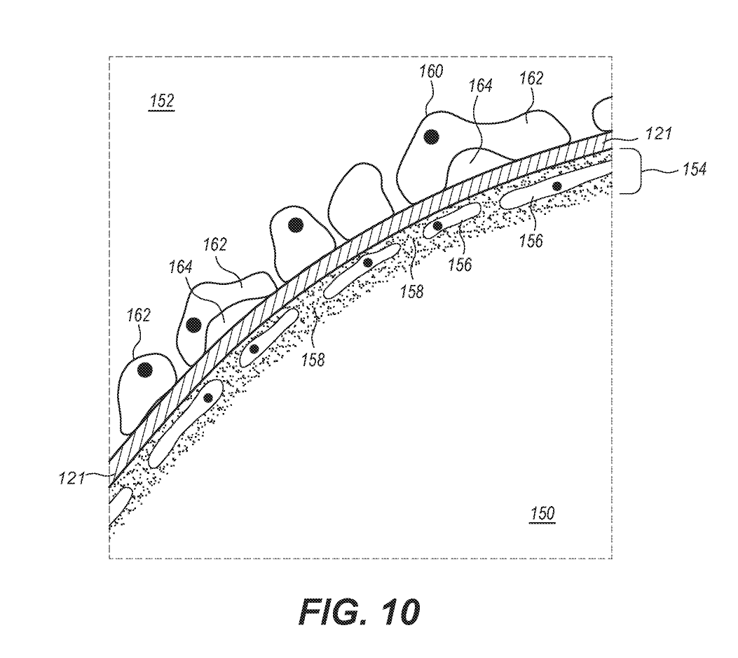

The invention finds a variety of uses, for example, as three dimensional biological engineering glomerular cell co-culture model systems (FIGS. 6, 7 and 8) for the study of glomerular system function and cell physiology, in response to varying cell culture parameters or varying biological engineering variables (via equipment parameter alterations). The invention also provides tools to study the effects of drug treatment (FIG. 9) on the glomerular filtration model system function and the cell physiology of vascular endothelial cells and podocyte cells in an ex vivo environment that more closely mimics the in vivo renal (glomerular) environment compared to static cell culture systems previously described. The invention also finds use as a model system for the study of renal disease, specifically, for diseases having etiology in the malfunction of the glomerular filtration step and diseases arising from aberrant physiology of the vascular endothelial cells and/or podocyte cells. By extension, the invention finds use in the study of drug effects on the glomerular filtration function, drug-induced cellular toxicity in these two cell types, and provides a model system for the prediction of efficacy of select drugs in the treatment of glomerular disease. This model system is also adaptable in order to screen and identify potential new drug candidates that potentially have the ability to restore or improve renal glomerular function in disease states.

Aspects of the invention are best understood from the appended drawings, as described below.

### II. Modified HFBR Systems

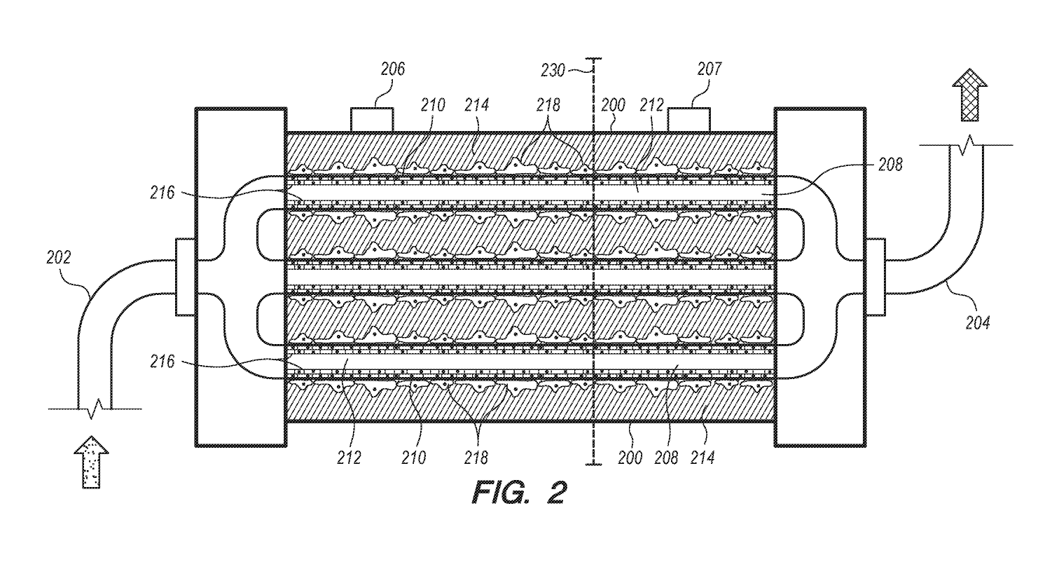

The invention incorporates a modified hollow fiber bioreactor design. The invention is best understood by review of the various embodiments shown in the drawings. FIG. 2 provides a schematic of a modified hollow fiber bioreactor cartridge unit of the invention. The cartridge comprises a cartridge wall 200 that forms an enclosed housing. The bioreactors of the invention use a plurality of semi-permeable hollow fibers 208, open at both ends, made from of porous synthetic material that forms the fiber walls 210. For illustration purposes, only three hollow fibers are shown in the schematic, although a plurality of many dozens, hundreds or thousands of such fibers within one cartridge are contemplated.

The hollow fibers 208 pass through a cartridge space enclosed by a cartridge wall 200 that forms an enclosed housing. Within the cartridge housing is at least one, and preferably a plurality of many hollow fibers, open at both ends, having semi-permeable walls 210 and an interior luminal space 212. The bioreactor is characterized by two partitioned spaces, i.e., two spatially-separated spaces or compartments. The first of these partitioned spaces is the luminal spaces 212 in the interior of the hollow fibers 208, through which culture medium passes in a directional flow from one end of the cartridge to the opposite end of the cartridge. Cell culture medium or whole blood enters the cartridge through an inlet port 202 at one end, shown by the stippled arrow. The cell culture medium or whole blood travels the length of the fiber and in a directional flow and exits the cartridge housing at the outlet port 204 on the opposite end of the cartridge, shown by a cross hatched arrow. The second partitioned space is the extra-capillary space (ECS) 214 that is defined as the space bounded by the interior surface of the cartridge housing wall 200 and the exterior surface of the hollow fiber 210.

Water and various soluble molecular and protein components of the cell culture medium or whole blood pass through the semi-permeable walls 210 of the fibers to enter the extra-capillary space 214. Glomerulus-derived vascular endothelial cells 216 are adhered on the luminal surface within the hollow fibers 208, and podocyte cells 218 are cultured on the exterior surface of the hollow fibers 208 within the extra-capillary space 214. The extra-capillary space 214 can be accessed, sampled or fed using ports 206, 207 located in the cartridge housing wall 200.

As used herein, the expression “in fluid communication” refers to a system where two or more reservoirs or partitioned spaces are coupled to each other in series by an unobstructed path of fluid, for example, in a circuit of culture medium, where the fluid path is not obstructed by a permeable or semi-permeable wall or membrane, for example, the porous walls of the hollow fibers of the co-culture systems of the invention.

As used herein, the expression “partitioned spaces” synonymously refers to “spatially separated” or “compartmentalized” spaces that lie in distinct domains bounded by the physical structures of the hollow fiber bioreactor cartridge. One of these physical structures is the semi-permeable walls 210 of the hollow fibers 208 which permit the flow of water and various solutes between the partitioned spaces. These partitioned spaces restrict the physical intermingling of the co-cultured cells. That is to say, the cell bodies of the two cell types do not contact each other. However, the partitioned spaces are in diffusible communication with each other via the liquid and solutes that can pass through the semi-permeable partition and travel between the partitioned spaces. That is to say, the luminal space of the hollow fibers and the extra-capillary space are in diffusible signal communication.

As used herein, the expression “in diffusible communication” or “in diffusible signal communication” refers to two partitioned spaces that are separated by a porous, semi-permeable barrier that prevents the migration of cells between the two compartments, but where the semi-permeable barrier permits the two-way exchange of smaller molecules between the partitioned spaces.

A semi-permeable barrier of any suitably porous material can be selected, and is customized for the intended use. For example, a semi-permeable barrier with a porosity that permits the exchange of small molecules such as dissolved carbohydrates, salt ions, amino acids and small polypeptides can be used, and conversely, that same barrier will prevent the migration of whole cells, such as the co-cultured adherent cells, red blood cells or large polypeptides exceeding a certain molecular weight. In some embodiments, a semi-permeable barrier that will prevent the free diffusion of albumin is preferred in order to mimic normal in vivo glomerular function. In other embodiments, semi-permeable barriers having larger porosity can be used, for example, with porosities that permit the diffusion of albumin, or red blood cells, in order to mimic various renal disease conditions.

Porous barrier materials that can support the growth of adherent cells is preferred, but materials that by themselves do not support adherent cell growth can be employed by coating or modifying the surfaces to promote cell attachment and growth, as well as to generate a natural, naturally secreted or artificial glomerular basement membrane (GBM).

The bioreactors of the invention are populated with two cell types, each in a distinct spatially limited (i.e., partitioned) area of the bioreactor. Adherent glomerulus-derived vascular endothelial cells are seeded onto the fiber interior luminal surface, thereby modeling the vascular endothelium that exists in the glomerulus capillaries. Podocyte cells are seeded in the extra-capillary space and adhere to the outside surface of the fibers. The co-culture is established using any suitable cell culture medium and culture growth conditions that are known to one of skill in the art to support the growth and maintenance of these particular cell types.

The adherent glomerulus-derived vascular endothelial cells and the podocyte cells are maintained in physically distinct partitions in the bioreactor. The podocyte cells are cultured in the extra-capillary space on the outside surface of the fibers. The actual cell bodies that make up the two cell populations in the co-culture presumably do not physically intermingle and do not come into physical contact because they are separated by the fiber wall barrier. Although the fiber wall is porous and semi-permeable, the pores in the fiber wall are too small in diameter to permit the passage of whole endothelial cells or whole podocyte cells. This is in contrast to some other types of co-culture systems which require physical contact between the co-cultured cell populations. However, in the present invention, even though the vascular endothelial cells and the podocyte cells remain in their respective physical compartments without intermingling, these cell populations are in “diffusible communication” by secreted diffusible factors (such as peptides, small proteins or non-protein signaling molecules) that are small enough to cross the porous fiber walls. These factors produced by one cell type can traverse the fiber wall and influence the behavior of the cell population on the opposite side of the fiber wall.

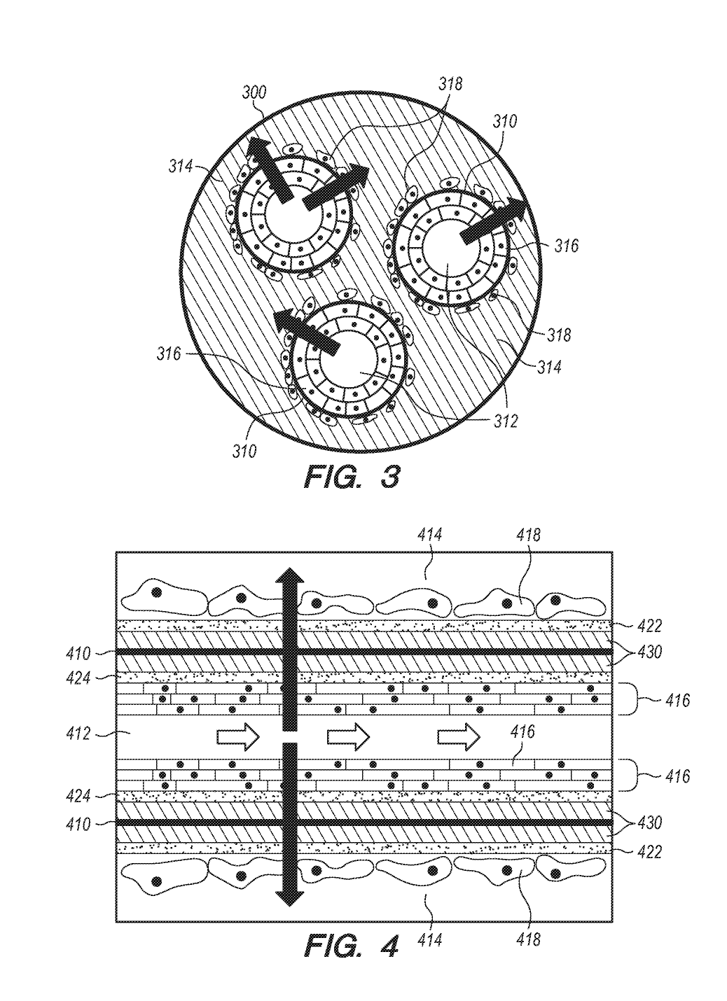

FIG. 3 shows a schematic of a hypothetical lateral transect of the hollow fiber bioreactor cartridge unit shown in FIG. 2, thereby illustrating generalized aspects of the invention. The lateral transect is shown as transect line 230 in FIG. 2. For illustration purposes, only three hollow fibers are shown in the transect view of FIG. 3. The drawing is not to scale. As shown in FIG. 3, a bioreactor cartridge of the invention comprises a cartridge wall 300 that forms an enclosed housing. The bioreactors of the invention use a plurality of semi-permeable hollow fibers made from walls of porous synthetic material 310. The hollow fibers pass through a cartridge space enclosed by the cartridge wall 300. The bioreactor is characterized by two partitioned spaces, i.e., (i) the luminal spaces 312 in the interior of the hollow fibers, through which culture medium flows, and (ii) the extra-capillary space (ECS) 314 that is defined as the space bounded by the interior surface of the cartridge housing wall 300 and the exterior surface of the hollow fiber 310. Water and various soluble molecular and protein components of the culture medium or whole blood that are below the filter molecular weight cut off (MWCO) pass through the semi-permeable walls 310 of the fibers, shown by the filled arrows 320, to enter the extra-capillary space 314. Glomerulus-derived vascular endothelial cells 316 are adhered on the luminal surface within the hollow fibers, and podocyte cells 318 are cultured on the exterior surface of the hollow fibers within the extra-capillary space 314.

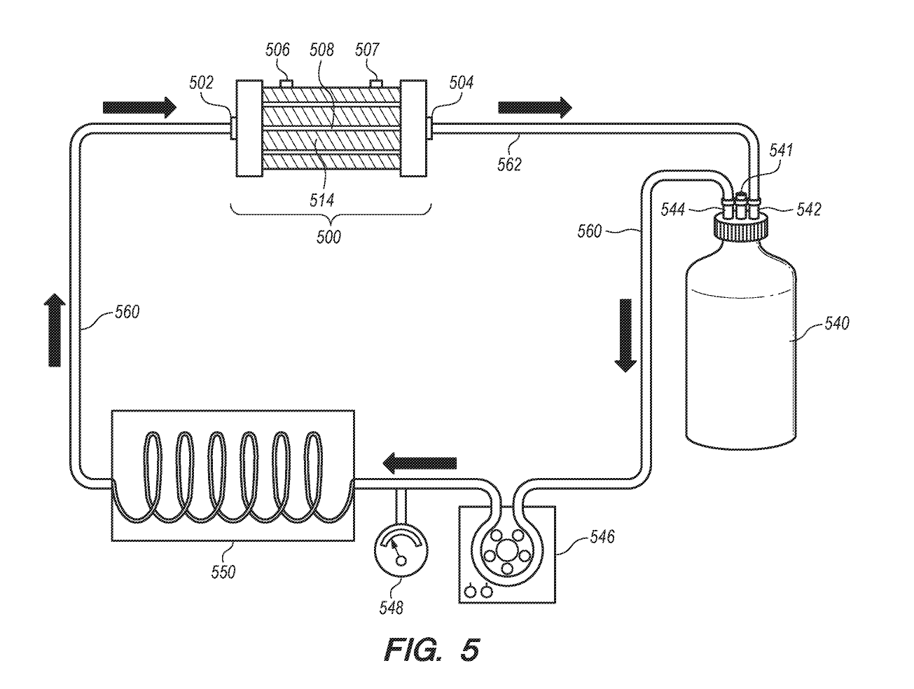

FIG. 4 provides a generalized schematic showing a hypothetical longitudinal transect of the bioreactor cartridge unit shown in FIG. 2. For illustration purposes, only one hollow fiber is shown in the transect, although a plurality of 10, 20, many dozens, hundreds or thousands of such fibers within one cartridge can be used. FIG. 4 is not drawn to scale. As shown in FIG. 4, the bioreactor cartridges of the invention incorporate a plurality of semi-permeable hollow fibers made from walls of porous synthetic material 410 which pass through the cartridge interior space. That is to say, the semi-permeable walls permit the free diffusion of molecules in soluble phase that are smaller than the defined size of the pores in the fiber wall. Any desired pore size, expressed alternatively as a molecular weight cut-off value (MWCO), can be chosen. For example, a MWCO consistent with normal kidney physiology at the interface of the glomerulus and Bowman's capsule can be used. Alternatively, a MWCO that mimics filtration leakage in glomerular disease can be utilized.

The bioreactor is characterized by two partitioned spaces, namely, (i) the luminal spaces 412 in the interior of the hollow fibers, through which culture medium or whole blood passes in a directional flow from one end of the cartridge to the opposite end of the cartridge, indicated by the empty arrows, and (ii) the extra-capillary space (ECS) 414 that is bounded by the interior surface of the cartridge housing (cartridge housing not shown in FIG. 4) and the exterior surface of the hollow fiber 410. Water and various soluble components of the culture medium or whole blood pass through the semi-permeable walls 410 of the fibers, shown by the solid arrows, to fill the extra-capillary space 414. During the co-culture establishment process, the metabolic waste products return to the fiber lumen and exit the retentate port. In the glomerular cell co-culture model systems, the molecules and proteins below the MWCO pass through the ECS and out the permeate port. Glomerulus-derived vascular endothelial cells 416 are adhered on the luminal surface within the hollow fibers, and podocyte cells 418 are cultured on the exterior surface of the hollow fibers within the extra-capillary space 414.

Also depicted in FIG. 4, optional coatings of a natural or artificial extracellular matrix material 430 can be applied prior to cell inoculation to the interior surface of the hollow fiber (i.e., the surface that lines the fiber lumen), the exterior surface of the hollow fiber, or both. Either the same or different ECM materials can be applied to the inside of the fibers and outside of the fibers.

This natural or artificial extracellular matrix material aids in cell attachment, cell growth, cell differentiation and maintenance of the cell culture. The natural or artificial extracellular matrix material is applied to the fiber surface or surfaces during the manufacturing process of the fiber and bioreactor cartridge, or immediately prior to inoculation of the fiber lumen and extra-capillary spaces with the respective cell types, depending on the type of ECM that is used. A variety of extracellular matrix materials can be used, as known to one of skill in the art. The extracellular matrix material that coats the interior surface of the fiber (i.e., the fiber lumen) can be the same or different from the extracellular matrix material that coats the exterior surface of the fiber in the extra-capillary space.

It is contemplated that the partitioned cell co-culture systems of the invention mimic the in vivo architecture of the glomerular filtration apparatus, including the glomerulus vasculature with endothelial cells lining the capillaries, and basement membrane deposition. As a result, the cultured adherent vascular endothelial cells attached to the interior walls of the luminal space and the adherent podocyte cells attached to the exterior surface of the fiber walls within the extra-capillary space will be expected in some embodiments to deposit basement membrane material onto their respective growth surfaces as occurs in the in vivo environment. As shown in FIG. 4, basement membrane material 424 can be optionally deposited/secreted by the cultured vascular endothelial cells on the interior surface of the fiber (i.e., on the walls of the fiber lumen). Similarly, basement membrane material 422 can be optionally deposited/secreted by the cultured podocyte cells on the exterior surface of the hollow fibers in the extra-capillary space. However, basement membrane material 422 and 424 may displace the coatings shown in 430.

The basement membrane material that is deposited/secreted by the vascular endothelial cells may be the same or different as the basement membrane material that is deposited by the podocyte cells. Notwithstanding the presence or absence of basement membrane material deposited by either the cultured vascular endothelial cells, the cultured podocyte cells, or both, it is not intended that the invention be limited in any regard by the presence or absence of basement membrane material deposited by these cell types. It is not intended that the invention be limited in regard to any theorized mechanisms of action or molecular physiology of the cultured cells.

### III. System Configurations and Apparatus

The invention provides modified hollow fiber bioreactor designs that can be used in a variety of configurations. These apparatus and systems of the invention are optimized for the co-culture of (i) adherent glomerulus-derived vascular endothelial cells on the interior surface of the fiber luminal space, and (ii) podocyte cells on the outside surface of the hollow fibers in the extra-capillary space.

The systems and apparatus of the invention have two partitioned spaces that are in diffusible communication. An extra-capillary space is defined as the space bounded by the interior surface of the cartridge housing and the exterior surface of the hollow fiber.

In the cell co-culture establishment phase, the cell co-culture hollow fiber bioreactor (HFBR) is initially configured to operate in batch mode with full (100%) retentate recycle, for example, as shown in FIG. 5. In this system, the cell culture medium is circulated from the feed (central) reservoir 540 through the luminal space 508 of the HFBR 500. Here, culture medium carrying nutrients and oxygen nourish the endothelial cells on the fiber lumen surface and then diffuse across the semi-permeable hollow fiber membrane to provide nutrients and oxygen to the podocyte cells on the outer hollow fiber surface. Spent media containing carbon dioxide and waste products (i.e. lactic acid) diffuse back into the hollow fiber lumen and are removed by the bulk flow of culture medium into the feed reservoir 540. The cellular metabolic waste products are significantly diluted in the feed reservoir 540 compared to the concentrations adherent cells are exposed to in a static culture system. Once the glucose levels are depleted to a sufficient level, the feed reservoir 540 is replaced by a reservoir of fresh culture medium.

The invention also provides three dimensional cell culture system configurations for modeling in vivo glomerular filtration that takes place in the renal corpuscle using whole blood as the media. See, for example, FIGS. 6-8. In some aspects, the invention provides tangential bulk flow filtration cell culture systems that mimic the in vivo glomerular filtration process. These model systems of the invention can incorporate hollow fiber bioreactor apparatus for the partitioned co-culture of glomerulus-derived vascular endothelial cells and podocyte cells in a manner that creates a three dimensional ex vivo model of the glomerular architecture. The culture systems of the invention have components that are analogous to the structure and function of the various components of the in vivo glomerular filtration apparatus that generates the glomerular filtrate.

In still other aspects, the invention provides methods and apparatus for generating an ex vivo post-filtration fluid (i.e., the permeate) that is analogous to in vivo glomerular filtrate. This whole blood based cell co-culture glomerular model system permeate of the invention finds use in further studies that model kidney proximal tubule function and proximal tubule cell physiology.

Some embodiments of the present invention described herein utilize hollow fiber bioreactor equipment manufactured by FiberCell® Systems, Inc. (Frederick, Md.). However, it is not intended that the invention be limited to bioreactor designs available from this one manufacturer, as equivalent designs using identical engineering principles are available from a variety of other manufacturers. See, for example, apparatus available from Spectrum Laboratories, including the CELLMAX® capillary cell culture systems (Rancho Dominguez, Calif.); Cellco, Incorporated (Germantown, Md.); Biovest International (Minneapolis, Minn.), General Electric Company doing business as GE Healthcare Life Sciences (Buckinghamshire, U.K.) and C3 Cell Culture Company (Minneapolis, Minn.).

The present disclosure provides a variety embodiments of the invention. However, it is appreciated by one of skill in the art that the full scope of the invention is not limited by the particular embodiments recited herein. One of skill recognizes that the scope of the invention encompasses additional designs not specifically recited herein, where those additional designs are extrapolated from the ample description and principles that are provided in the present disclosure.
A) Batch Mode Configuration with Full Retentate Recycle and No Permeate Flow Stream; Cell Co-Culture Establishment System

In some embodiments, the perfusion bioreactors of the invention are configured as batch mode systems where the retentate is recycled to the feed reservoir in a culture system circuit. One possible configuration of a continuously recycling bioreactor circuit of the invention is shown in FIG. 5. In this configuration, a hollow fiber bioreactor cartridge 500 is fed via a feed port 502 connected to a feed line 560 that is fluidly connected to a culture medium reservoir 540 via an outlet port 544 on the reservoir 540. Culture medium is drawn from the culture medium reservoir 540 by the action of an in-line pump 546 that drives the directional flow and perfusion of culture medium. Examples of such a pump can be a peristaltic type pump or a pulsatile type pump. Any number of pumps can be deployed along the culture medium circuit to promote the directional flow of culture medium through the circuit and to maintain suitable positive pressures in the hollow fiber luminal spaces 508. One or more in-line pressure gauge 548 (or any type of suitable pressure sensor) installed in any suitable locations along the circuit is used to monitor the pressure in the system. An in-line oxygenation mechanism 550 for oxygenation of the culture medium and removal of dissolved carbon dioxide can also be employed in the system.

The culture medium that is fed into the hollow fiber cartridge 500 through the feed port 502 passes through the hollow fiber luminal spaces 508 and emerges at the collection port 504, where the medium is sent through a collection line 562 (also termed a recycle line) back to the culture medium reservoir 540 through an inlet port 542 in the reservoir. The captured culture medium in the culture medium reservoir 540 is recirculated, i.e., recycled, back into the cartridge 500 through the feed line 560 and reservoir port 544. A small volume of culture medium permeate passes across the hollow fiber walls into the extra-capillary space 514, but this permeate is not collected in the configuration shown in FIG. 5. The culture medium reservoir 540 can further contain one or more ventilation filters 541 for air pressure equilibration. The direction of culture medium flow is indicated by arrows.

This configuration shown in FIG. 5 can be used, for example, for the initial establishment of the co-culture, that is to say, immediately after inoculation of the bioreactor for the initial growth phase of the cells in the culture. This particular configuration does not continuously divert and segregate a permeate flow that would hypothetically pass from the luminal space of the hollow fibers 508 and into the extra-capillary space 514.

The configuration shown in FIG. 5 or any other figure is not intended to be limiting, as it is apparent from the present description that the configuration shown in FIG. 5 can be readily modified, for example, with sampling ports (e.g., 506 and 507) at any desired points to facilitate removal of culture medium for testing, and further, can be configured to sample, divert and/or segregate the retentate flow and/or permeate flow from the cartridge 500.
B) Batch Mode Glomerular Cell Co-Culture Model System with Permeate Capture and Diafiltration

FIG. 6 shows another batch mode system configuration, but in contrast to the system of FIG. 5, the system of FIG. 6 incorporates a permeate flow capture and the addition of a diafiltrate. This co-culture system shown in FIG. 6 operates in batch mode, but with the addition of a permeate flow that captures the components from the whole blood or other culture medium that passes from the fiber luminal space, through the semipermeable walls of the hollow fibers and into the extra-capillary space. Thus, this system more closely models the in vivo glomerular function, where the permeate flow is analogous to the in vivo glomerular filtrate.

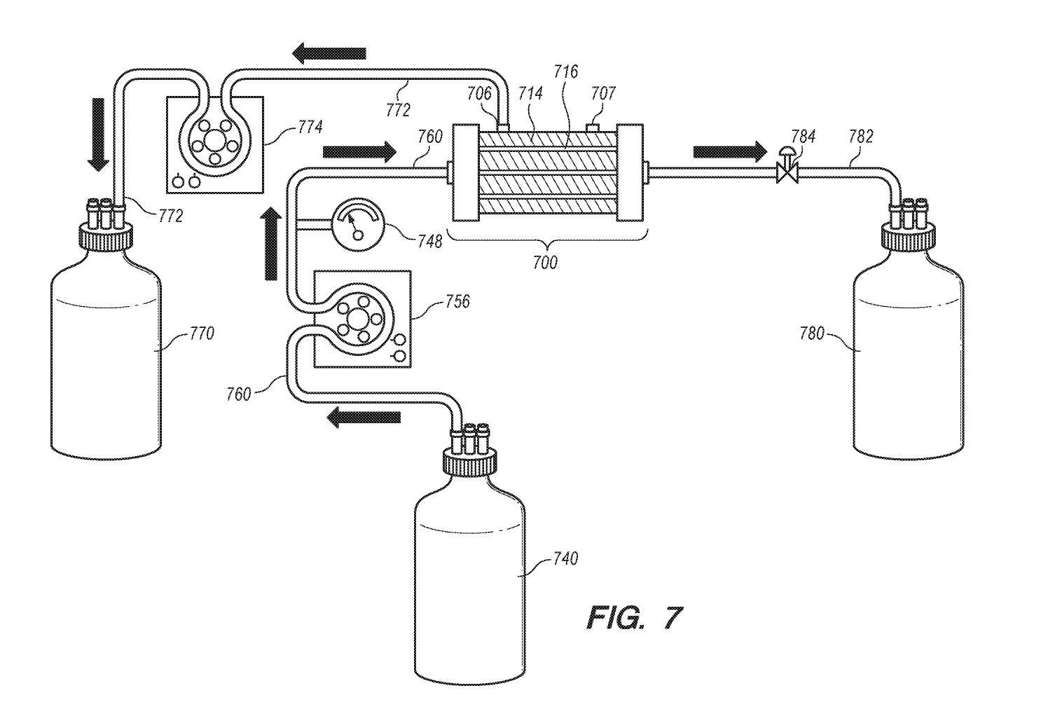

In the FIG. 6 configuration, the components of the blood fraction that are below the filter membrane MWCO are pumped from a diafiltration reservoir to ensure constant feed conditions in terms of blood component concentration. This configuration is an alternative way to configure the system to prevent the feed reservoir from having non-physiological concentrations of blood proteins, cells etc. There may be times when, due to aspects of disease conditions or the presence of drugs, retentate recirculation is undesirable and this is not a suitable configuration. However, this system will be more cost-effective than the continuous mode system shown in FIG. 7.

In this particular embodiment shown in FIG. 6, whole blood is drawn through a feed line 660 by the action of an in-line pump 646, from the whole blood feed reservoir 640 to the bioreactor cartridge 600, which is in fluid communication with both the upstream and downstream fiber luminal spaces 608. The liquid is pumped through the entire length of the luminal spaces of the fibers, in a tangential directional flow. The pump can be a peristaltic type pump or a pulsatile type pump or any other type of suitable pump. Any number of pumps can be deployed along the medium circuit to promote the directional flow of medium and maintain positive pressure in the hollow fiber luminal spaces. One or more in-line pressure gauge 648 (or any type of suitable pressure sensor) can be installed in any suitable locations along the circuit. An in-line oxygenation unit 650 is also employed.

This particular embodiment is described in the scenario that whole blood is used as the culture medium, although this configuration is not limited for the use of whole blood as culture medium. When the apparatus is configured to model normal glomerular filtration function, that is to say, using parameters that mimic glomerular health, the retentate, which contains whole cells (i.e. red blood cells and white blood cells) and proteins above the MWCO of the membrane (i.e. albumin in conditions of glomerular health), is recirculated via the retentate line 662 to the feed reservoir 640. The flow of the retentate can be regulated by a suitable in-line valve 661 in the retentate line 662. The media is then redelivered to the bioreactor cartridge 600 by a feedline 660. Additionally, the serum blood fraction that contains the proteins and molecules below the MWCO of the membrane is pumped from the diafiltration reservoir 680 through the diafiltration line 682 into the feed reservoir 640 at the same flow rate that the permeate is removed.

The whole blood passes through the fiber luminal spaces in a tangential directional flow, where whole blood components below the MWCO are forced through the semi-permeable walls of the hollow fibers 608 by a positive pressure (created by at least one pump 646) within the lumen to flow into the extra-capillary space 614 and generate the permeate flow. In contrast to the system shown in FIG. 5, a permeate flow stream is captured from the extra-capillary space 614 and that permeate is collected in a permeate collection reservoir 670 connected to and in fluid communication with the extra-capillary space 614 via a port 606 and permeate collection line 672. Additional ports 607 can be installed in the cartridge wall for sampling of the permeate flow from the ECS 614.

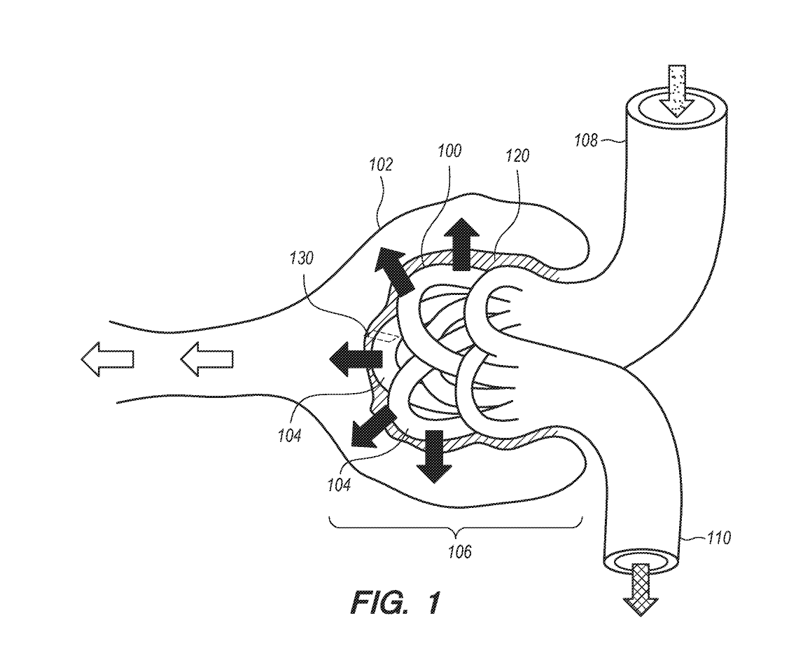

In this embodiment, the permeate that passes into the extracellular space 614 is analogous to the in vivo glomerular filtrate that passes into the Bowman's capsule space (see FIG. 1 at 102). The retentate flow is analogous to the blood flow that exits the glomerulus in the renal corpuscle through the efferent arteriole (see FIG. 1 at 110).
C) Continuous Mode Configuration Glomerular Cell Co-Culture Model System with Retentate Capture and Permeate Capture

FIG. 7 provides a schematic showing a tangential flow filtration bioreactor system of the invention that uses a continuous infusion of fresh whole blood or blood product. The apparatus contains a bioreactor cartridge 700 that is fed through a feed line 760 from a feed reservoir 740 that is a source of fresh whole blood or blood product. The delivery of whole blood is driven by at least one in-line pump 756. Culture medium is passed along the length of the hollow fibers in the cartridge 700 in a tangential directional flow. The fraction of the whole blood medium that passes through the full length of the hollow fiber luminal spaces is termed the retentate fraction, which is collected in a retentate collection reservoir 780 that is in fluid communication with the downstream fiber luminal spaces via a retentate collection line 782. The flow of the retentate can be regulated by a suitable in-line valve 784 in the retentate line 782. In this embodiment, the retentate fraction does not return to the culture medium feed reservoir 740.

The fraction of whole blood/blood product medium that passes through the fiber walls into the extra-capillary space 714, termed the permeate flow, is collected in the permeate collection reservoir 770, which is connected to and in fluid communication with the extra-capillary space 714 via a cartridge permeate port 706 and permeate line 772. Additional ports 707 can be installed in the cartridge wall for facilitating sampling of the permeate flow from the ECS 714.

In this embodiment, the whole blood feed reservoir 740 will contain only fresh whole blood that has not been exposed to the cell co-culture in the bioreactor cartridge. Thus, the fresh whole blood medium that enters the bioreactor cartridge 700 through the inlet port (also termed a feed port) will ultimately be partitioned into two fractions; first, the retentate fraction that passes through the full length of the fiber luminal space, and second, the permeate fraction that is forced through the semi-permeable walls of the hollow fibers and into the extra-capillary space.

A feed pump 756 is shown installed in the feed line 760, although one or more supplemental pumps can also be installed, for example, with a permeate pump 774 in the permeate line 772. Pressure gauges 748 or sensors of any type can be installed in multiple locations in the system, including the feed line 760, retentate line 782, or in the permeate line 772. An oxygenation module can optionally be incorporated.

In this embodiment as well as other embodiments, sampling ports can be installed at any suitable location in the culture medium circuit, including in any of the flow lines. This has the advantage that the sampling of the retentate flow and the permeate flow can be done in real time, so that solute concentrations of these fractions can be monitored for changes over a course of time.

In some embodiments, it is advantageous to segregate the permeate flow and the retentate flow so that those flows, if they are e.g. affected by disease conditions or drug treatment, they do not contain unrepresentative feed components upon recirculation. Additionally, the captured and segregated permeate and retentate streams can be independently analyzed and quantitated for the purpose of characterizing the filtration process that is occurring in the bioreactor cartridge. For example, the filtration flow rates can be measured, and/or various components of the flow streams can be qualitatively detected or quantitatively measured as markers indicative of integrity of the filtration activity occurring when the whole blood passes from the fiber lumen to the extra-capillary space. In some embodiments, markers include molecular markers, such as serum albumin or total protein. Cell-specific markers indicative of cellular health or pathology include, for example, synaptopodin and WT-1 as markers for podocyte physiology, and CD31 as a marker for glomerular endothelial cells.
D) Batch Mode Configuration Glomerular Cell Co-Culture Model System with Retentate Recycle and Permeate Recycle

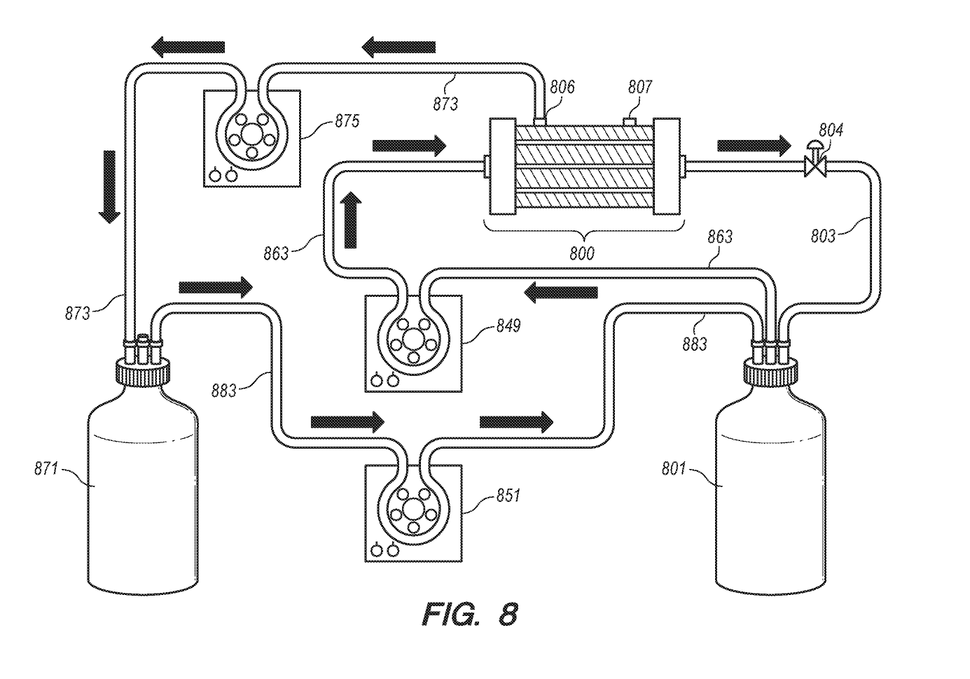

FIG. 8 provides a schematic of a variation of the batch mode cell co-culture system of FIG. 6. The system of FIG. 8 shows an alternative configuration of a batch mode system where both the permeate stream and the retentate stream are fully recycled into a feed reservoir that supplies the feed line to the bioreactor cartridge. In contrast, the system shown in FIG. 6 recycles only the retentate stream.

The system shown in FIG. 8 depicts a batch mode operation of the flow system, in contrast to the continuous feed mode depicted in FIG. 7. As previously mentioned, in this batch mode operation shown in FIG. 8, both the permeate stream and the retentate stream are recycled into the feed reservoir 801 that supplies the bioreactor cartridge 800. In this embodiment, the bioreactor cartridge 800 is fed with a line 863 from a central/feed reservoir 801. That central reservoir 801 is supplied from two sources, which are (i) the retentate stream that has passed through the bioreactor cartridge 800, through a retentate recycle line 803 and back into the central/feed reservoir 801; and (ii) the permeate stream that is captured initially in a permeate capture line 873 through an ECS port 806, collected in a permeate collection and recycling reservoir 871, then redirected back to the central/feed reservoir 801. The permeate capture flow can be assisted by an in-line permeate capture pump 875. Similarly, the flow from the permeate collection and recycling reservoir 871 to the central reservoir 801 through the recycle line 883 can be assisted by an in-line pump 851.

Pumps can be deployed in various locations, including a feed pump 849 installed in the feed line 863, a permeate capture pump 875 in the permeate capture line 873 and a permeate return pump 851 located in the permeate return line 883. Similarly, pressure sensors can be installed in multiple locations in the system, including upstream or downstream of pumps located in the feed line 863, retentate line 803, permeate capture line 873 and/or permeate return line 883. An oxygenation module is incorporated (not shown). One or more additional access port 807 to the ECS compartment can be installed. Any useful valve can be installed anywhere in the system; for example, the flow of the retentate back to the central feed reservoir 801 can be regulated by a suitable in-line control valve 804 in the retentate line 803.

This embodiment has the advantage of reducing costs compared to the continuous feed system of FIG. 7, which in contrast to FIG. 8, requires the continuous stream of fresh whole blood, which can be expensive. This embodiment as shown in FIG. 8 has the additional advantage of avoiding potential non-physiological elevated concentrations of protein components in the feed reservoir, as in FIG. 8 the retentate volume and permeate volume are remarried in the central/feed reservoir 801. This configuration can be used provided there are no issues with permeate recycle or retentate recycle, as may occur under disease conditions or with the use of drugs.
E) Continuous Mode Configuration Glomerular Cell Co-Culture Model System for Testing Drug Therapeutic Effects and Drug Toxicity

FIG. 9 provides a schematic of an alternative configuration of the partitioned glomerular cell co-culture model system shown in FIG. 7, where the system has been adapted for testing the potential therapeutic effects of drugs or for testing for drug toxicity.

In this configuration, a first drug reservoir 950 containing whole blood supplemented with a first drug is connected to and is in fluid communication with the feed reservoir 940. Delivery of the whole blood supplemented with the first drug to the feed reservoir 940 is via a supply line 952 driven by a first drug supply line pump 954. Similarly, a second drug reservoir 990 containing whole blood supplemented with a second drug is also connected to and is in fluid communication with the feed reservoir 940. Delivery of the whole blood supplemented with the second drug to the feed reservoir 940 is via a supply line 992 driven by a second drug supply line pump 994. A third reservoir termed the diluent reservoir 960 containing only whole blood (which acts as a diluent) is also connected to and is in fluid communication with the feed reservoir 940. Delivery of the diluent whole blood to the feed reservoir 940 is via a supply line 962 driven by a diluent supply line pump 964.

Any or all of the pumps 944, 954, 964, 974 and 994 can be any style of suitable pump, and further, can be programmable in any regard, for example, to deliver fluid volume at a designated flow rate, to turn on/off at any designated time or when any particular condition is achieved or desired, for example, to add a first drug or second drug, or to increase or decrease a drug concentration in the feed reservoir 940. Any other useful valves to regulate liquid flow can be installed elsewhere in the system, without restriction.

Whole blood supplemented with a first drug is pumped from a reservoir 950 either alone or in combination with whole blood containing a second drug, which is pumped from a second reservoir 990 into the feed reservoir 940 from lines 952 and 992, respectively. Reservoir 962 can be filled with whole blood without drugs (diluent), which is used to dilute the drug concentration in the feed reservoir 940.

The whole blood with or without drug(s) is pumped in a tangential directional flow from the feed reservoir to the HFBR 900 via feed line 942. The material that travels the full length of the fiber lumen, in a flow tangential to the membrane filter, and passes through to the retentate line 982 is e.g. collected in a retentate reservoir 980. The permeate passes through the semipermeable membrane under positive pressure and is collected in a permeate reservoir 970 after passing through a permeate flow line 972.

### IV. Hollow Fiber Specifications

The invention incorporates a modified hollow fiber bioreactor (HFBR) design resulting in two partitioned spaces in diffusible communication. The bioreactors of the invention incorporate porous hollow fibers, also termed capillaries or membranes, resulting in a luminal space in the interior of the hollow fibers, and an extra-capillary space (ECS) that exists outside of the fiber wall membranes.

The HFBR systems consist generally of a cartridge containing many cylindrical porous fibers, also termed capillaries, e.g., 10 to many hundreds or thousands of fibers, of uniform length (e.g., approximately 10 cm in length or longer) and small diameter (e.g., approximately 700 μm inner diameter, although smaller or wider inner diameters can be used).

The fibers are constructed of a porous semi-permeable material characterized by a molecular weight cut off (MWCO) value expressed in molecular mass units of kilodaltons (kDa), which represents an approximate nominal (lower limit) in molecular size for a dissolved solute that will be retained to greater than 90% by the hollow fiber membrane (EMD Millipore. 2016. “Pore size or NMWL (MWCO),” Life Science Research). The porous wall permits the passage of molecules smaller than the MWCO, including nutritional components (e.g., glucose and dissolved oxygen) and drugs into the ECS from the luminal space into the extra-cellular space, and permits the reverse flow of metabolic waste (e.g., lactate) out of the ECS into the luminal media stream. The porous fiber walls block the passage of whole cells, cell debris and proteins with molecular sizes above the MWCO. Alternatively, the fiber porosity can be expressed in an actual pore dimension, for example, a pore diameter, or average pore diameter, expressed in microns (micrometers; μm), instead of a molecular weight cut off value.

The retention of a protein by any given porous barrier is not just dependent on the molecular weight of that protein or the physical dimension of the pores in the hollow fiber walls. Retention is affected by protein characteristics such as size, net charge, shape (determined by secondary, tertiary and quaternary structure, as well as pH and ionic conditions) relative hydrophobic and hydrophilic nature of the protein, concentration in sample, external functional groups, extent of denaturation, operating conditions and Stokes radius (Pall. 2016. “Choosing the correct MWCO.” Ultrafiltration fundamentals; Caret, R. L. et., 1995. “Foundations of inorganic, organic, and biological chemistry.” Wm. C. Brown Publishers, Boston). Furthermore, whether a given protein will pass through a fiber membrane pore will be impacted by the membrane chemistry. For example, fiber walls constructed of hydrophobic materials will tend to exclude a protein that has outward facing hydrophilic side chains or hydrophilic post-translational modifications, even though the properly folded protein is sufficiently small in diameter to theoretically pass through those pores.

The table below provides an approximate relationship between molecular weight cut off values (in kDa) and pore diameter size (expressed in microns) (Aisimo Corporation LTD (London, UK), 2013).

TABLE 1
Relating kilodaltons to microns.

| MWCO | Pore Diameter |
|---|---|
| kilodalton (kDa) | microns (micrometers; μm) |
| 1,000 | 0.1 |
| 500 | 0.02 |
| 200 | 0.01 |
| 50 | 0.004 |
| 10 | 0.0025 |
| 5 | 0.0015 |

The table above is only a guideline to compare MWCO values with pore diameter values. Consistent classification of a porosity rating of a membrane between manufacturers is challenging because various manufacturers utilize different criteria to score the porosity of the membranes. In designating the MWCO porosity of the hollow fiber walls, the effectiveness of the retention of the molecules through the fiber wall can be expressed in various ways. For example, if a membrane is rated as having a 5 kDa MWCO, that could mean a hypothetical protein will demonstrate 50% retention by the membrane. More typically, a membrane rated as having 5 kDa MWCO that could mean a hypothetical protein will demonstrate 90% retention, which is considered a “useful retention” for proteins that are the size of that hypothetical protein and larger, by that membrane. For example, if a fiber wall has a rating of 5 kDa MWCO, that fiber wall may have a useful retention property for proteins with 25 kDa molecular weight or larger. In some embodiments, as a general rule, a membrane with a molecular weight cut off value is selected that is three to six times smaller than the molecular weight of the protein of interest that is to be retained by the membrane (Pall. 2016. “Choosing the correct MWCO.” Ultrafiltration fundamentals).

A suitable MWCO restriction can be selected to mimic normal renal (glomerular) cell physiology. Most notably, a fiber wall having a molecular weight cut-off (MWCO) value that will effectively exclude albumin protein (i.e., will retain at least 90% of the albumin protein in a solution) can be used. This restriction on diffusion in the culture system will mimic the retention of albumin in the glomerular capillaries that occurs in vivo.

For example, in some embodiments, a fiber with a suitably small MWCO is selected that will retain whole cells and polypeptides larger than about 65 kilodaltons (kDa) from crossing the fiber wall, but permit the passage of smaller proteins and molecules such as glucose, oxygen and cellular waste products such as lactic acid. The 65 kDa molecular weight restriction threshold is significant in that it will mimic the in vivo blood filtration that occurs at the interface of the glomerulus and Bowman's capsule which, in healthy cells, excludes circulating blood components, most notably, albumin having a molecular weight of 65 kDa. A 65 kDa size restriction will also prevent the passage of still larger moieties into the Bowman's capsule, such as immunoglobulins and red blood cells. Any porosity wall that will prevent the passage of albumin across the fiber wall but permit the flow of nutritional components is contemplated to find use with the invention, because this range of porosities is theorized to mimic the in vivo glomerulus—Bowman's capsule interface filtration apparatus in normal cells in the absence of renal (glomerular) disease.

In the present glomerular co-culture model system, whole blood is used as a medium, which contains serum albumin. A hollow fiber membrane can be chosen (e.g. 10-20 kDA MWCO) that will effectively exclude passage of that protein albumin into the permeate flow.

Alternatively still, the MWCO value of a fiber wall membrane can be custom manufactured to any desired value at the time of manufacture using techniques known to one of skill in the art.

The ex-vivo modelling of various renal (glomerular) diseases using the compositions, apparatus and methods of the invention is achieved by using hollow fibers having a MWCO values larger than the MWCO values that retain or effectively retain albumin. In such embodiments, the parameters of effective retention of at least 90% albumin retention are not satisfied, and significant quantities of albumin protein pass from the fiber luminal spaces into the extra-capillary space and appear in the permeate flow. This scenario mimics in vivo aspects of various renal (glomerular) diseases, most notably, renal diseases that arise because there is loss of integrity of the filtration apparatus and defective production of glomerular filtrate.

Furthermore, grades of severity of disease in glomerular filtration can be modeled by using hollow fibers with increasing large MWCO values. A size restriction can be selected to mimic glomerular disease state. For example, in some embodiments, a fiber with a MWCO value is selected that does not effectively retain albumin from crossing the fiber wall, e.g., retains less than 90% of the albumin protein. Alternatively, diseased cells (vascular endothelial cells ad podocyte cells carrying genetic defects), aberrant extra-cellular matrix materials, and/or missing or defective GBM components, alone or in combination with hollow fiber membrane materials that model non-physiological filtration properties, for example a membrane having a 0.1 μm MWCO with diseased glomerular endothelial and diseased podocyte cells, can result in an altered glomerular filtration membrane that mimics glomerular disease states.

The hollow fibers can be constructed of any suitable materials as known in the art, most notably, synthetic materials that support growth of adherent cultured mammalian cells. The hollow fibers can also be constructed from any suitable naturally occurring materials or artificial organic materials, or hybrids of any combination of synthetic and non-synthetic (naturally occurring) materials and/or organic or inorganic materials (see for example Slater et al., 2011. “An in vitro model of the glomerular capillary wall using electrospun collagen nanofibres in a bioartificial composite basement membrane,” PLoS ONE 6(6): e20802). Examples of materials finding use in the construction of the hollow fibers includes, but not limited to, hydrophilic polysulfone, hydrophobic polyethylene, cellulosic fibers or polyvinylidene difluoride (PVDF).

### V. Extracellular Matrix and Glomerular Basement Membrane

Ideally, the hollow fibers used in the bioreactors of the invention are constructed of a material known to support growth of adherent cultured mammalian cells. However, other materials that do not support or only weakly support the culture of adherent mammalian cells can also be used, where those fiber materials can be pre-coated, for example, with a natural or artificial extracellular matrix (ECM) material or any other material or molecular moiety that beneficially modifies the growth properties of the surface. For example, coatings of a natural or artificial extracellular matrix material or other types of coatings can permit or improve cell attachment, accelerate cell growth and/or induce proper cell differentiation of the cultured cells.

In some embodiments, cells finding use with the invention, e.g., glomerulus-derived vascular endothelial cells and podocyte cells, can be cultured directly on the surface of the hollow fiber, that is to say, on the interior surface of the hollow fiber and on the exterior surface of the hollow fiber, respectively, in the absence of any ECM coating(s) applied to the fiber surface(s) prior to inoculation of the cells into the respective surfaces.

In other embodiments, cells finding use with the invention, e.g., glomerulus-derived vascular endothelial cells and podocyte cells, can be cultured on the surface of the hollow fiber, that is to say, on the interior surface of the hollow fiber and on the exterior surface of the hollow fiber, respectively, where the fiber surface(s) have been precoated with any suitable ECM material prior to inoculation of the cells into the respective surfaces.

As used herein, cultured cells such as glomerulus-derived vascular endothelial cells and podocyte cells, are cultured “associated with” either the interior surface or exterior surface of the hollow fiber (respectively), where “associated with” includes either in the absence or presence of any precoated ECM material. Thus, the cultured cells are associated with their respective surfaces of the hollow fiber in the alternative scenarios of no ECM coating or with ECM coating. As used herein, the expression “associated with” a surface of the hollow fiber can alternatively mean adhered to the surface of the hollow fiber in the absence of ECM or in the presence of ECM.

In various embodiments, the extracellular matrix material can be deposited on the outside of the hollow fiber, on the inner luminal walls of the fiber, or on both the inner and outer surfaces of the fibers. In some embodiments, one ECM composition is used on the outside of the hollow fiber, and a different ECM composition is used on the inside of the hollow fiber.

In some embodiments, the exact composition of the natural or artificial extracellular matrix material to be coated on the growth surface is customizable for the particular cell type that is being seeded onto the surface. In some embodiments, ECM proteins are naturally secreted by the cells and deposited onto the surface. In some embodiments, the natural or artificial ECM promotes the attachment of proteins or other molecules (e.g. gelatin, fibronectin, collagen, or other proteins contained in fetal bovine serum) to the fiber wall, thereby promoting cell attachment to these surfaces.

In some embodiments, the ECM that is added to the fiber surface has a charge function in defining the filtration properties of the fiber wall that occurs when components of the culture medium or whole blood pass from the luminal spaces to the ECS to form a permeate in the ECS. That is to say, the presence of ECM affects the filtration properties of the fiber wall.

A variety of ECM products are known to one of skill in the art. For example, the following ECM products and cel adhesion molecules find use with the invention, including but not limited to, fibrin, fibrinogen, fibronectin, ProNectin®-F (Sigma-Aldrich®), Matrigel® matrix and BioCoat™ coatings (Corning® Life Sciences), various collagen species, for example type IV collagen, laminin proteins, synthetic polymers such as glycolic acid, agrin, heparin sulfate proteoglycan (perlecan) and nidogen.

The in vivo interface between the glomerular capillaries and the Bowman's capsule is a complex layered structure that regulates the filtration of blood from the glomerular capillaries to form glomerular filtrate. Lying between the endothelial cell layer on the luminal surface of the glomerular capillary wall and the podocyte cells attached to the surface facing the Bowman's space, is a basement membrane, termed the “glomerular basement membrane” or GBM. In vivo, this basement membrane is deposited by the combined activity of the endothelial cells and podocyte cells.

The glomerular basement membrane is an especially thick basement membrane that contributes to the filtration barrier at the glomerulus—Bowman's capsule interface. The mature GBM consists primarily of laminin-521 (α5β2γ1), type IV collagen α3α4α5(IV), nidogens-1 and -2, and agrin (a heparin sulfate proteoglycan). Type IV collagen and laminins are the most significant of the major GBM components. These GBM components are deposited by the adjacent vascular endothelial cells and the adjacent podocyte cells.

Glomerular endothelial cells and the podocyte cells secrete different protein components in the GBM, and furthermore, the deposited protein components change as a function of time and cell maturity. It is theorized (Abrahamson, D. 2012. “Role of the Podocyte and Glomerular Endothelium in Building the GBM,” Semin Nephrol., 32 (4): 342-349) that juvenile isoforms have the likely advantage of enabling the glomerular endothelial cells and the podocyte cells to be spread more easily on the glomerular basement membrane (GBM) surface.

Laminins LM-111 and LM-411 which are derived/secreted from immature podocytes, and collagen IV α1α2α1 is derived/secreted by immature endothelial cells. It is theorized that these early isoforms of laminin and collagen IV GBM proteins bind to the integrins of the podocytes (α3β1) and the integrins of the glomerular endothelial cells (α1β1 and α2β1), respectively (Abrahamson, D. 2012. “Role of the Podocyte (and Glomerular Endothelium) in Building the GBM,” Semin Nephrol., 32 (4): 342-349; Scott, R. and S. Quaggin. 2016. “The cell biology of renal filtration,” J. Cell Biol, 209 (2): 199-210; Miner, J. 2012. “The glomerular basement membrane,” Exp Cell Res, 318 (9): 973-978.)

The cells will secrete the adult isoforms and substitute the early isoforms with the adult LM-521 and the adult collagen α3α4α5IV. The laminar isoform substitution requires the presence of both podocytes and glomerular endothelial cells, as LM-521 is secreted by both, while the adult collagen α3α4α5IV is secreted by podocyte cells alone (Abrahamson, D. 2012. “Role of the Podocyte (and Glomerular Endothelium) in Building the GBM,” Semin Nephrol., 32 (4): 342-349; Scott, R. and S. Quaggin. 2016. “The cell biology of renal filtration,” J. Cell Biol, 209 (2): 199-210).

Alternatively, artificial deposition of any one or more of the components normally found in vivo in the GMB can be used to coat either one or both of the hollow fiber surfaces in the hollow fiber bioreactors of the invention prior to seeding the two cell types. It is contemplated that such coatings will further mimic the in vivo filtration architecture and can induce the cells to display physiology consistent with the in vivo state. Any combination of immature or mature cell secretion profiles can be used in the coatings, and furthermore, asymmetric deposition of these components can be made onto the luminal surface and the extra-capillary surface to optimize attachment, growth and differentiation of the two respective cell types that attach to these two different surfaces. For example, the outer surface of the fibers of the HFBR can be precoated with laminins LM-111 and LM-411, which are secreted from immature podocytes or LM521. The inner fiber lumen can be precoated with collagen IV α1α2α1, which is normally derived/secreted by immature endothelial cells or collagen α3α4α5IV. As the cells mature, this can result in a natural or naturally secreted (in more mature form) GBM.

Extracellular materials to be used as coatings in the bioreactors of the invention are available from many manufacturers. See, for example, Neuromics (Edina, Minn.) which manufacturers laminin-1, human collagen IV and other extra-cellular protein supplements; AMS Biotechnology (Europe) Limited, doing business as AMSBIO LLC (Cambridge, Mass.), which manufactures a wide variety extracellular matrix supplements, including AMSBIO Cultrex® basement membrane extracts (BME), collagen preparations, laminin preparations, fibronectin preparations and AMSBIO MAPTrix™, which is a recombinant, animal-free mussel adhesive protein (MAP)-based chemically defined extracellular matrix biomimetic. AMSBIO MAPTrix™-L laminin and MAPTrix™-C collagen products are also available. See also ABCAM PLC (Cambridge, Mass.) which manufactures natural human collagen IV protein (Product No. ab7536) and other reagents finding use as ECM materials. In addition, custom production of natural adult and juvenile GBM proteins may be available.

It is contemplated that the glomerular endothelial cells and the podocyte cells that are seeded onto their respective faces of the hollow fibers in the culture apparatus may also deposit basement membrane components onto the porous walls of the fibers, contributing to the filtering capacity of the fiber walls. In some embodiments of the invention, hollow fibers having wall porosities larger than might be expected to mimic the in vivo environment can be used, where the filtering function of the model system is achieved or fine-tuned after the cultured endothelial cells and/or podocyte cells deposit GBM components onto the porous fiber walls. In that scenario, the fiber wall acts as a scaffold for the endothelial cells and/or podocyte cells to deposit the materials that result in a proper functioning GBM. This is critical, since according to the literature (Sanyal, S. 2014. “Culture and assay systems used for 3D cell culture,” review article, Corning Life Sciences application note 245, document no. CLS-AC-AN-245 (publ. Corning Life Sciences)), maintaining the mechanical and chemical properties of the GBM in a dynamic manner from seeding to cell growth and development is necessary to accurately model the ECM/GBM. Also, the glomerular cells sense the mechanical properties of their environment through their integrins, and cell adhesion through GBM proteins in three-dimensional cell cultures results in more physiologically representative (compared to two dimensional systems) cellular responses.

Similarly, it is contemplated that filtering characteristics of the model systems of the invention that mimic the in vivo glomerular filtration system can be fine tuned to mimic in vivo properties, or can be modified to mimic disease states, through the use of larger MWCO hollow fiber filtration membranes, by deposition of suitable natural or artificial coating or natural or artificial extracellular matrix onto either or both sides of the porous walls of the capillaries prior to inoculating with the two cell types. The use of glomerular endothelial cells and/or podocyte cells derived from human patients showing renal (glomerular) disease, or animal cells containing genetic defects resulting in or theorized to contribute to renal (glomerular) disease, or using whole blood from human patients showing renal (glomerular) disease as the culture medium, can contribute to building ex vivo model systems that closely mimic in vivo renal pathology and renal disease states.

Defects in the glomerular basement membrane result in aberrant filtration. Of the nine proteins found in the GBM, mutations in the genes encoding four of them are known to cause glomerular disease in humans as well as in mice. These are (Miner, 2012. “The glomerular basement membrane,” Exp. Cell Res., 318(9): 973978):

| Gene | Protein |
|---|---|
| LAMB2 | Laminin Subunit β2 |
| COL4A3 | Collagen Type IV α3 |
| COL4A4 | Collagen Type IV α4 |
| COL4A5 | Collagen Type IV α5 |

Mutation of a fifth gene (the Lama5 gene, encoding Laminin Subunit α5) in podocytes in mice causes proteinuria, nephrotic syndrome, and progression to renal (glomerular) failure. These results highlight the importance of the GBM for establishing and maintaining a properly functioning glomerular filtration barrier. Furthermore, this information can be used to construct glomerular cell co-culture model systems that mimic renal (glomerular) disease, for example, by seeding the bioreactors of the invention with endothelial and/or podocyte cell lines that carry these genetic defects.

### VI. Cells and Cell Culture Conditions

A variety of endothelial cells and podocyte cells find use with the invention to populate the hollow fiber bioreactor luminal and exterior surfaces, respectively. The cell lines that are used are not particularly limiting, and can be from any suitable source for glomerular-derived vascular endothelial cells and podocyte cells. In some embodiments, the cells that are cultured are primary cells, also termed primary cell cultures, which are derived from human or animal tissue. In other embodiments, finite cell cultures can be used, which are formed by the passaging of a primary cell culture. Such finite cell cultures will proliferate only for a limited number of passages. In still other embodiments, continuous cell lines are used, also termed established cell lines or immortalized cell lines. Continuous cell lines have the capacity for indefinite subculture. Continuous cell lines that are transformed cell lines also find use with the invention. (See Geraughty et. al., 2014. “Guidelines for the use of cell lines in biomedical research,” Br J Cancer, 111(6): 1021-1046).

Cells isolated from or derived from any mammalian species find use with the invention. For example, endothelial cells and podocyte cells that are mammalian cells, i.e. porcine cells, marsupial cells, rodent cells, mouse cells, rat cells, primate cells and human cells all find use with the invention. Cells of human origin find particular use with the invention. When cells from one mammalian source are identified for use as either endothelial cells or podocyte cells, it is preferable to use cells derived from that same mammalian species as the source for the second cell population. For example, it is preferable to co-culture a mouse established podocyte cell line with a mouse endothelial cell line in the co-culture partitions.

In some aspects, cells that are cultured in the bioreactor systems of the invention are intended to model “normal” or healthy cell physiology. For example, primary cell cultures that are derived from healthy human subjects or from mouse tissue that do not display any renal pathology can be used in the systems of the invention. Similarly, some continuous/established cells lines are also suitable to model normal renal (glomerular) physiology.

In alternative embodiments, cells that are cultured in the bioreactor systems of the invention are intended to model renal disease and/or abnormal cell physiology. For example, primary cell cultures that are derived from human subjects that display renal disease can be used in the systems of the invention. Similarly, some continuous cells lines are also suitable to model renal pathology. Also finding use in this aspect of the invention are genetically modified cells that contain aberration(s) in one or more gene known to be involved in regulating renal function. Genetically modified cells can include transgenic cell lines and/or cells derived from transgenic animals containing genetic modifications, such as knock-out or knock-in type genetic modifications.

Endothelial cells finding use with the invention are glomerular-derived vascular endothelial cells. That is to say, they are endothelial cells derived from the capillaries of the glomerulus. In other preferred aspects, the glomerular-derived vascular endothelial cells are derived from glomerular microvascular endothelial tissue. Primary human glomerular microvascular endothelial cells can be derived from decapsulated glomeruli isolated from normal human kidney cortical tissue, as known in the art. Continuous/established endothelial cell lines are also available for use with the invention.

Podocyte cells are visceral epithelia in origin and face the urinary space in the Bowman's capsule. These cells are closely associated with the outer aspect of the glomerular basement membrane. Podocyte cells finding use with the invention can come from (or be derived from) any source, including primary cell cultures, or continuous/established cell lines. In some embodiments, podocyte cells are derived from mammalian urine. In some aspects of the invention, podocyte cells are selected from mammalian podocyte cells, i.e. porcine podocyte cells, rodent podocyte cells, mouse podocyte cells, rat podocyte cells, primate podocyte cells and human podocyte cells.

A variety of sources for the co-cultured glomerular-derived vascular endothelial cells and podocyte cells finding use with the invention are also known. See, for example but not limited to: Bruggeman et al., 2012. “A cell culture system for the structure and hydrogel properties of basement membranes; Application to capillary walls,” Cell. Mol. Bioeng., 5(2): 194-204; Byron et al., 2014. “Glomerular cell cross-talk influences composition and assembly of extracellular matrix,” J Am Soc Nephrol., 25(5): 953-966; Li et al., 2016. “Three-dimensional podocyte-endothelial cell co-cultures: Assembly, validation, and application to drug testing and intercellular signaling studies,” European Journal of Pharmaceutical Sciences 86:1-12 (epub. Feb. 23, 2016); Slater et al., 2011. “An in vitro model of the glomerular capillary wall using electrospun collagen nanofibres in a bioartificial composite basement membrane,” PLoS ONE 6(6): e20802; Sun et al. (2013), “Glomerular endothelial cell injury and damage precedes that of podocytes in adriamycin-induced nephropathy,” PLoS ONE 8(1): e55027; and Rastaldi, M and M. Li. “Method for the three-dimensional co-culture of podocytes and endothelial cells and relative in vitro co-culture system”. Patent Application Publication, Pub no.: US 2013.0177929 A1, Jul. 11, 2013.

Additional sources for glomerular-derived vascular endothelial cells finding use with the invention are also known. See, for example but not limited to: Rops et al., 2004. “Isolation and characterization of conditionally immortalized mouse glomerular endothelial cell lines,” Kidney International, 66: 2193-2201; Satchell et al., 2006. “Conditionally immortalized human glomerular endothelial cells expressing fenestrations in response to VEGF,” Kidney International, 69: 1633-1640. See also glomerular endothelial cells distributed by Cell Systems Corporation (Kirkland, Wash.), for example, human glomerular microvascular endothelial cells having Applied Cell Biology Research Institute certificate number ACBRI-128.

Additional sources for podocyte cells and podocyte cell culture techniques finding use with the invention are also known. See, for example but not limited to: Kabgani et al., 2012. “Primary Cultures of Glomerular Parietal Epithelial Cells or Podocytes with Proven Origin,” PLoS ONE 7(4): 1-12, e34907; Krtil et al., 2007. “Culture Methods of Glomerular Podocytes,” Kidney Blood Press Res., 30: 162-174; Chittiprol et al., 2011. “Marker expression, behaviors, and responses vary in different lines of conditionally immortalized cultured podocytes,” Am J Physiol Renal Physiol., 301(3): F660-671; Chuang et al., 2013. “Capturing the in vivo molecular signature of the podocyte,” Kidney International, 83(6): 986-988; Ni et al., 2012. “Podocyte culture: tricks of the trade,” Nephrology 17: 525-531; Shankland et al., 2007. “Podocytes in culture: past, present, and future,” Kidney International 72(1): 26-36; and Sakairi et al., 2010. “Conditionally immortalized human podocyte cell lines established from urine,” Am J Physiol Renal Physiol., 298(3): F557-567.

Genetically modified cells that contain aberration(s) such as mutations or other types of transmissible genetic modification in one or more gene known to be involved in regulating renal (glomerular) function find use with the invention, and in particular, in co-culture systems that model renal glomerular pathology. Genetically modified cells can include transgenic cell lines and/or cells lines derived from transgenic animals containing genetic modifications, such as knock-out or knock-in type genetic modifications, or express a transgene of interest relevant to the study of renal physiology.

Genetically modified cells finding use with the invention to model renal (glomerular) pathology can come from (or be derived from) transgenic animals, for example, transgenic mice. For example, mice carrying one or more transgene of interest, or have mutations at endogenous loci that impact kidney function, find particular use.

Cells carrying one or more genetic anomaly, such as a mutation of interest, find use with the invention, where the genetic anomaly is known to correlate with renal disease, for example, in humans or mice. Such mutations can have arisen naturally, for example, mutations that exist in cell lines (primary or continuous) derived from organisms having renal disease. Cell lines derived from human patients with renal disease find particular use in this regard. Alternatively, the genetic anomaly can have been intentionally engineered, such as in an engineered cell line, or derived from a transgenic animal that stably contains artificially manipulated genetic material.

Genetic anomalies or and engineered genetic backgrounds find use in the model systems of the invention as they pertain to both glomerular-derived vascular endothelial cells and podocyte cells. Cells derived from humans or animals that display renal disease phenotypes, and therefore are suspected of carrying a genetic anomaly, also find use with the invention. Cells derived from humans or animals that display renal (glomerular) disease phenotypes find use with the invention regardless of whether or not the disease phenotype is a genetic trait.

One of skill in the art is familiar with protocols, culture conditions, nutrient media formulations and other reagents for the culture of animal cells, including glomerular-derived vascular endothelial cells and podocyte cells. Cells can be cultured in conventional defined nutrient media unmodified or modified as necessary, and using culture conditions known in the art. Numerous sources for protocols and media formulations for the culture of animal cells include, for example, Freshney, R. I. (2016). “Culture of Animal Cells, a Manual of Basic Technique and Specialized Applications”, 7th Edition, Wiley-Blackwell. See also the references cited herein, and also other scientific literature, cell repository information, and manufacturers' literature providing recommended culture media formulations and culture conditions for particular bioreactor apparatus and particular cell lines. In some embodiments, particularly when glomerular cell co-culture systems are being simulated, whole blood, including blood from diseased patients (when disease conditions are being simulated) can be used in the glomerular cell co-culture model system, typically after a period of cell establishment using traditional defined culture medium.

### VII. Modeling of Renal (Glomerular) Function

The invention provides three dimensional cell culture systems for modeling in vivo glomerular filtration that takes place in the glomerulus-Bowman's capsule interface. The culture systems of the invention have components that are analogous to the structure and function of the various components of the in vivo glomerular filtration apparatus, and further, present advantages of dynamic co-culture model systems.

In some aspects, cells that are cultured in the bioreactor systems of the invention are intended to model “normal” or healthy cell physiology. In alternative embodiments, cells that are cultured in the bioreactor systems of the invention are intended to model renal disease and/or abnormal cell physiology.

The cell culture systems of the invention provide improvements in model systems that mimic the in vivo glomerular architecture and the in vivo juxtaposition of glomerular vascular endothelial cells and podocyte cells, compared to static cell culture systems, thereby enabling more accurate modeling of the glomerular filtration function. The cell culture systems of the invention enable specialized cell differentiation/function due to the in vivo-like spatial orientation and three dimensional architecture of the growth environment.

The hollow fibers incorporated into the cartridge mimic the selectivity of the in vivo glomerular filtration apparatus. Fiber walls having a molecular weight cut-off (MWCO) value that will exclude albumin protein (e.g., will retain at least 90% of the albumin protein in a solution) can be used. This restriction on diffusion in the culture system will mimic the retention of albumin in the glomerular capillaries that occurs in vivo. The filtration properties of the hollow fiber can be further modulated by coating the fibers with one or more extracellular matrix protein, and alternatively, or in addition, the cells that are cultured on the interior and exterior surfaces of the fibers will also modify the filtration properties of the porous wall.

There are a number of advantages of the hollow fiber perfusion-type bioreactor culture system of the invention over traditional static cell culture methods for the co-culture of glomerulus-derived vascular endothelial cells and podocyte cells, which are realized by the present invention. These include the following:

The fluid mechanics and tangential flow of the model systems of the invention generate an interstitial flow that more accurately mimics in vivo conditions compared to static culture systems. In the present systems, endothelial cells are exposed to more physiological levels of chronic shear stress. Endothelial cells under chronic shear stress behave differently compared to cells under static culture conditions, e.g., cells under shear stress lie flat in a monolayer whose orientation is determined by the direction of flow of the culture medium and form tight junctions. Hence, the cultured endothelial cells of the invention are grown in a physiological environment that is closer to that found in vivo in the glomerular capillaries (Cadwell, J. 2004. “New developments in hollow fiber cell culture.” American Biotechnology Laboratory).

Factors secreted by endothelial cells inside the hollow fiber are able to migrate and affect podocyte cells outside of the fiber (i.e., attached to the outside of the hollow fibers in the extra-capillary space), and vice versa, thus permitting signaling between the two cell types which is likely required for proper cell function and mimicking in-vivo conditions.

The permeate flow stream and the retentate flow stream are easily sampled to obtain quantitative readouts indicating status of the fluid streams in the glomerular model system. This includes, for example but not limited to, measuring the permeability of the endothelium/fiber-wall GBM/podocyte structure to albumin, which can be determined by measuring the albumin concentrations in the permeate stream and retentate stream in a continuous flow mode or batch flow mode system. Further, a sieving coefficient can be calculated from these quantitative measurements. Alternatively, any molecular marker indicative of cell health, cell toxicity or disease can be conveniently monitored in the permeate flow or retentate flow using the apparatus of the invention.

The bioreactors of the invention provide improved model systems for the study of glomerular function, and as a result, also provide a more physiologically accurate model system for studying drug migration (mass transfer) across the glomerular filtration apparatus. This occurs due to the fact that cells are exposed to a dynamic tangential flow, and unlike most perfusion cultures, there is a filtration function during the glomerular model system simulations of this invention. In addition, the bioreactor model systems of the invention provide improved systems for the study of human pharmacodynamics by better modelling drug uptake and exposure to metabolic products due to a dynamic (versus static) drug exposure through tangential flow, along with exposure to a filtration function. This results in a better pharmacodynamics assessment. The use of whole blood to deliver a drug to the glomerular cell co-culture system more accurately simulates drug bioavailability. The model systems of the invention also enable a more physiologically accurate exposure to different drug concentrations, different drug dosing intervals, different drug concentration gradients (changes in concentration with respect to time), and different drug combinations than in current glomerular model systems, due to both the tangential flow and the filtration function of the invention.

The secretion of glomerular endothelial cell products, such as endothelin-1 can be studied as a function of shear rate. Shear stress (ss=μ4Q/πr³, where μ=viscosity, Q=flow rate and r=fiber radius and ss=shear stress, tangential) is proportional to shear rate. Shear rate is defined as the gradient of velocity i.e. the rate of change of velocity between adjacent fluid (i.e. blood) layers.

In the model systems of the invention, factors secreted by the podocytes, such as vascular endothelial growth factor (VEGF) and angiopoietin-1 (Ang-1) can bind to their receptors on the glomerular endothelial cells, VEGFR2 and Tie2, respectively, as they would in vivo. See Schrijvers et al., 2004. “The role of vascular endothelial growth factor (VEGF) in renal pathophysiology,” Kidney International, 65: 2003-2017. This enables the study of the effects of VEGF and Ang-1 in controlling endothelial cell behavior. Similarly, factors secreted by the GECs, such as endothelin-1 (ET-1), can be studied in in-vivo like conditions to determine their effects on the contraction of podocyte actin cytoskeleton and loss of nephrin (slit diaphragm protein). This is valuable since it is known that ET-1 plays a critical role in both normal physiological and pathophysiological conditions. See Dhaun, N. et al., 2012. “Endothelin-1 and the kidney—beyond bp,” British Journal of Pharmacology, 167: 720-731. In addition, cross-talk between endothelial cells and podocyte cells is crucial for obtaining a more in vivo representative GBM. See Byron, A. 2013. “Glomerular cell cross-talk influences composition and assembly of extracellular matrix,” J Am Soc Nephrol., 25(5): 953-966.

The pumps installed to control the flow of cell culture medium or whole blood into the cartridge feed port, secondary pumps that can assist in the regulating the flow of permeate from the extra-capillary space, and optional control valves placed in-line i.e. in the retentate flow line can control the pressures of the feed flow (P_{F}), the extra-capillary space that supplies the permeate flow (P_{P}) and the retentate pressure (P_{R}), respectively. This pressure regulation system allows the modeling of pressures analogous to the in viva afferent arteriole pressures and efferent arteriole pressures, as well as pressures in the Bowman's capsule, or at least the net filtration pressure. This model system also simulates a pressure and a flow rate analogous to the glomerular capillary pressure [(P_{F}+P_{R})/2] and glomerular filtration rate (GFR). The use of a pulsatile pumps can further mimic hemodynamic flow in the glomerulus.

The permeate flow in the system apparatus can be used to model the in vivo flow of glomerular filtrate that empties into the proximal tubule. The invention provides methods and apparatus for generating an ex vivo post-filtration fluid (i.e., the permeate) that is analogous to the in vivo glomerular filtrate. This cell culture permeate of the invention finds use in studies involving modeling the function of the kidney proximal tubule. In the case of drug toxicity studies, in addition to toxic effects of drugs on the glomerulus, the permeate flow from the glomerulus co-culture model system obtained during drug toxicity experiments can be subsequently exposed to proximal tubule cells to determine downstream toxicity.

The co-culture systems of the invention can be used to assess drug toxicity on endothelial cells and podocyte cells growing in the co-culture, thereby providing a barometer for predicting in vivo renal pathology and toxic effects in patients in the event that the drug is administered to a patient.

The co-culture systems of the invention can be used to assess the ability of a drug to improve the cell physiological status or glomerular system function of either endothelial cells and podocyte cells growing in the co-culture. That is to say, the invention can be used to assess the ability of a drug to improve or restore the filtering capacity of the co-culture model system. By extension, the ability of a drug to improve or restore the filtering capacity of the co-culture model system of the invention is predictive of whether or not that drug is a candidate drug that might show efficacy in improving or restoring renal (glomerular) function in viva.

Since mesangial cells are not present themselves in the HFBR model, vasodilation and vasoconstriction can be represented by equipment size change (hollow fiber/tubing size increase or decrease) and efferent arteriole pressure can be altered by altering retentate control valve setting. The advantage of this model in studying the effects of mesangial contraction (which causes glomerular capillary contraction, see Ghayur, M., et al. 2008. “Contractility of the renal glomerulus and mesangial cells: lingering doubts and strategies for the future.” Med Hypotheses Res. 4 (1): 1-9.) is that the hollow fiber (analogous to the glomerular capillary) size can be reduced without affecting the feed tubing (analogous to the afferent arteriole), thus uncoupling the effects of the glomerular capillary analogue contraction from the effects of the afferent arteriole analogue contraction (which impacts the renal blood flow rate). This is not possible using other investigative tools that use agonists that cause contraction of both the afferent arteriole and the glomerular capillary.

### VIII. Modeling of Renal (Glomerular) Disease

The effects of pathological conditions that affect the vasculature can be modeled by altering the equipment size or processing conditions. The effects of vasoconstriction can be determined by constricting the tubing through the use of a retentate control valve or decreasing the size of the tubing/hollow fibers (since one of the ways that the vasomotor region of the brain controls blood pressure is via the diameter of the blood vessel). Conversely, the effects of vasodilation can be modelled through the use of larger size tubing/hollow fibers, or opening the retentate control valve.

Similarly, aspects of glomerlar disease that alter glomerular filter permeability can be modeled by incorporating alternative capillary fiber materials having different molecular weight cut off (MWCO) ratings (which is a function of the pore size in the fiber walls) or different natural or artificial/synthetic coatings on the fiber wall. Furthermore, the hydrophobic or hydrophilic properties of the fiber wall can be modified by the use of different hollow fiber materials, or application of various compounds that are used to coat the fiber walls prior to cell inoculation. Direct comparisons of the effects of membrane permeability can be made by using model systems that are identical in all other respects.

The systems of the invention can be configured to model various glomerular disease conditions. For example, the co-cultures can be established using cells derived from diseased patients, for example human urinary podocyte cell cultures from patients with focal segmental glomerulosclerosis (FSGS), can be cultured on the exterior fiber walls in the extra-capillary space. Alternatively, using cells containing genetic defects in proteins that regulate renal (glomerular) function, such as mutations in genes that encode components of the glomerular basement membrane or using cells transformed with genes for glomerular disease is feasible.

In another example, healthy cells in a bioreactor culture can be exposed to hyperglycemic blood (e.g., in excess of about 10 millimoles per liter (mmol/L), or in excess of about 180 milligrams per deciliter (mg/dL). This treatment over time can induce the effects of diabetic nephropathy. Also, elevated blood glucose levels can increase flowrate of blood into the kidney (see Zhang, C. et al., 2014. Journal of Diabetes Research, Article ID 953742), which can be mimicked by increasing the feed flowrate to the hollow fiber bioreactor. Following creation of the disease condition, the cells can be exposed to various drug treatments to observe the effects of those drug treatments.

Healthy podocyte cells and endothelial cells can be co-cultured, and after establishment, exposed to antibodies specific to components of the glomerular basement membrane to mimic conditions of a damaged basement membrane. This type of experimental system can also incorporate whole blood or blood product.

Endothelin-1, which is secreted by the glomerular endothelial cells, has been found to play a role in both physiological and pathophysiological processes. See Dhaun, N. et al., 2012. “Endothelin-1 and the kidney—beyond bp,” British Journal of Pharmacology, 167: 720-731. Using the co-culture systems of the invention, the effects of endothelin-1 blocking agents (e.g., the small molecule atrasentan, sold as Xinlay™ from Abbott Laboratories, Inc.) on damaged podocytes can be observed.

Proteinuria can be established by increasing pore size of the fiber wall, such that the changes in the culture permeate mimic the increased passage of protein in vivo through the glomerular filtration apparatus.

In other embodiments, whole blood from a patient showing symptoms of kidney (glomerular) disease, or having a kidney (glomerular) disease of defined etiology, can be utilized as the culture medium in the glomerular cell co-culture model system of the invention (i.e. FIGS. 6, 7, 8 and 9). The use of such a culture reagent can mimic a glomerular disease physiological state, thereby providing a useful model for the study of that disease.

### IX. Assessment of Model System Function

Historically, various physiological tests have been developed as standard measures of kidney function in health and disease. These tests can be adapted for use with the glomerular cell culture model systems of the present invention which model health and disease, especially with regard to glomerular function to generate the glomerular filtrate. These methods generally assess the state of kidney function and indicate that substantial loss of kidney function has already occurred. In the glomerular cell co-culture model systems of the invention, the permeate that passes from the whole blood in the fiber lumen to the extra-capillary space corresponds to the in vivo glomerular filtrate. Historical methodologies for assessing kidney function (more specifically, proper production of glomerular filtrate) that find use with the invention are listed below.

GLOMERULAR FILTRATION RATE (GFR). GFR is used to measure kidney function and the degree of renal pathology, and is related to the net filtration pressure (NFP) (Guyton, A. C and J. E. Hall. 2013. “Textbook of medical physiology—11^{th} ed.,” Elsevier-Saunders, New York). The normal GFR is ˜120-125 ml/min and is reduced during renal disease to less than 60 ml/min (National Institute of Health. 2016. “Glomerular filtration rate,” US Notional Library of Medicine). Permeate flowrates can be measured directly from the permeate tubing and are analogous to the glomerular filtration rate as the permeate is analogous to the glomerular filtrate. Proper scaling of the GFR can result in direct comparisons to the clinical values. The result can be normalized for the number of fibers (i.e. divided by the number of fibers) and scaled to compare to actual kidney GFR levels, or alternatively, the measured GFR analogue values (permeate flow rate measured directly) in a healthy model system can be compared to the measured permeate flow rate values in a diseased model system. Different permeate flowrates can indicate different stages of disease or recovery.

Proteinuria—

Proteinuria is an excess total serum protein in the urine, and is indicative of glomerular disease (Fauci et. al., 1998. Harrison's principles of internal medicine. McGraw-Hill, New York. p. 1508). The condition arises from abnormal permeability of the glomerular capillaries, thereby allowing passage of protein from the blood supply, into the glomerular filtrate and then into the proximal tubule. The model systems of the invention generate a bioreactor permeate, which can mimic the proteinuria condition by using hollow fibers with increased permeability to proteins or generally to other large molecules. This can be done by using fiber walls having larger molecular weight cut-off ratings, for example, using materials having MWCO values along with diseased cells, which increase filter permeability so that more proteins, including higher molecular weight proteins, are released into the permeate.

In humans, treatment for proteinuria is classified in one of three states characterized by the passage of total protein in urine in a 24-hour period. These are:
complete remission (CR)—proteinuria<0.3 g/24 hr
partial remission (PR)—proteinuria 0.3-2.0 g/24 hr
no response (NR)—proteinuria> or =2.0 g/24 hrs
   See Thompson et al., 2015. “Complete and partial remission as surrogate end points in membranous nephropathy,” J Am Soc Nephrol. 26(12): 2930-2937.

The permeate in the bioreactors of the invention are analogous to the in vivo glomerular filtrate, which is the initial step in the generation of urine. The total protein concentration in the bioreactor permeate provides a value analogous to a measurement for proteinuria. Again, this can be measured directly by analyzing the permeate under both diseased and healthy conditions, and comparing the result. Alternatively, if this value is normalized for the number of fibers and properly scaled, with the expected tubule reabsorbed fraction accounted for, the values can be compared directly to clinical values (urine amounts over a specified time).

More generally, a sieving coefficient for any given molecule in the cell culture medium can be calculated, which is the ratio of the concentration of the substance of interest in the permeate to the concentration in the retentate. A sieving coefficient in the model systems of the invention is determined as follows:

$$Sieving Coefficient of a molecule of Interest = \frac{concentration of the molecule in the permeate}{concentration of the molecule in the retentate}$$

ALBUMINURIA. Albuminuria is the presence of albumin in the urine, indicative of kidney disease (National Institute of Health. 2016b. “Kidney tests,” US National Library of Medicine). The glomerular cell co-culture systems of the invention produce a permeate, which is analogous to the glomerular filtrate in vivo. The albumin concentration in the model systems of the invention can be measured directly in the permeate in both diseased model systems and healthy model systems, and the results compared. Also, a comparison to clinical values can take place with normalization for the number of fibers, appropriate scaling and accounting for the tubular reabsorption that takes place. The sieving coefficient is the ratio of the concentration of the substance of interest (i.e. albumin) in the permeate to the substance of interest (i.e. albumin) in the retentate. Again, this sieving coefficient can be measured in both diseased and healthy conditions, and the results compared.

Hematuria.

Hematuria is the abnormal presence of blood in the urine (National Institute of Health. 2016b. “Kidney tests,” US National Library of Medicine). For the case of disease, red blood cells in the permeate of the cell co-culture systems of the invention (measured as number of red blood cells/mL of culture permeate) is analogous to and can model (if normalized for the number of fibers and appropriately scaled) in vivo hematuria.

Creatinine.

Creatinine is a muscle metabolic waste product that is present in serum. In a healthy condition, creatinine is filtered (and removed) by the kidneys. However, if the kidneys are functionally compromised, this can result in increased creatinine levels in the serum (Guyton, A. C and J. E. Hall. 2013. “Textbook of medical physiology—11^{th} ed.,” Elsevier-Saunders, New York). Serum creatinine tests are used to determine the level of creatinine in the blood and estimates the kidney filtration function via the glomerular filtration rate. In this model system, whole blood will be used from healthy or diseased patients that should contain creatinine. The levels of creatinine in the retentate can be measured and compared in health versus disease, or the retentate creatinine values can be normalized for the number of fibers and appropriately scaled, in order to be used for GFR determination.

### Techniques for Evaluating Culture Systems

The physiological status of the co-culture systems of the invention can be evaluated by a wide variety of techniques. These techniques generally are characterizing the presence or absence of the two cell types that are co-cultured in the systems of the invention, or are looking directly at those cells using various microscopy techniques.

Microscopy.

A variety of microscopy techniques find use with the invention. Transmission electron microscopy (modified standard TEM), for example, that uses pre-fixation treatment with a cationic dye such as cupromeronic blue can enable visualization of the ESL (endothelial surface layer) ultrastructure. Scanning electron microscopy can be used to examine, for example, the pores of the fenestrae. The cells can be accessed by opening the fiber cartridge of the HFBR and removing the fibers without disturbing the cells. See Redmond et al., 1995. “Perfused transcapillary smooth muscle and endothelial cell co-culture—a novel in vitro model,” In Vitro Cell. Dev. Biol., 31: 601-609.

Cell Morphology.

Phase contrast microscopy can be used to examine cell morphology. Electron micrographs can be used to determine endothelial cell and podocyte injury, the latter for example, in focal segmental glomerulosclerosis (FSGS). Endothelial injury is indicated by subendothelial widening (SW) of the glomerular basement membrane (GBM), which is quantitated as the percentage of the length of the glomerular basement membrane showing subendothelial widening. Endothelial damage is also indicated by (i) a decrease of endothelium or its fenestrations, and (ii) endothelial swelling (Taneda et al., 2015. “Podocyte and endothelial injury in focal segmental glomerulosclerosis: an ultrastructural analysis,” Virchows Arch., 467(4): 449-458).

Podocyte injury can be indicated by (i) morphometric measurements of foot process width (FPW), which is inversely correlated with the presence of disease, or (ii) podocyte detachment (PD) from the basement membrane, which is quantitated as the percentage of basement membrane showing PD. See, for example, Taneda et al., 2015. “Podocyte and endothelial injury in focal segmental glomerulosclerosis: an ultrastructural analysis,” Virchows Arch., 467(4): 449-458.

Histological Alterations in Diabetes Mellitus.

Histological alterations are observed in diabetes mellitus, where hypertrophy of glomerular components and thickening of the basement membrane are seen. See Kumar, P. A., et al. 2014. “Molecular and cellular events mediating glomerular podocyte dysfunction and depletion in diabetes mellitus.” Frontiers in Endocrinology. 5: 1-10. Intact fibers can be removed from the bioreactor cartridge. The cells can be accessed by opening the fiber cartridge of the HFBR and removing the fibers without disturbing the cells. Fibers with the adherent cells attached can undergo immune-histochemical techniques to characterize morphological and histological features of the cells. Redmond et al., 1995. “Perfused transcapillary smooth muscle and endothelial cell co-culture—a novel in vitro model,” In Vitro Cell. Dev. Biol. Anim., 31: 601-609.

Cell Counts.

Absolute number of podocyte cells predicts glomerular function (Kumar, P. A., et al. 2014. “Molecular and cellular events mediating glomerular podocyte dysfunction and depletion in diabetes mellitus.” Frontiers in Endocrinology. 5: 1-10. Podocyte cell number goes down in response to toxins. Podocyte detachment is a measure of podocyte injury. Podocyte detachment can be measured in the co-culture model systems of the invention by analyzing podocyte counts in the culture permeate and retentate.

Apoptosis.

An apoptosis marker in endothelial cells is CD31, and an apoptosis marker in podocyte cells is synaptopodin. See Sun et al. (2013), “Glomerular endothelial cell injury and damage precedes that of podocytes in adriamycin-induced nephropathy,” PLoS ONE 8(1): e55027. Expression of these markers can be assayed in the cell co-cultures of the present invention as a measure of endothelial cell or podocyte cell apoptosis in co-culture.

Analytical Methods.

A variety of analytical methods find use with the current invention, in particular in the detection and/or quantification of various protein and nucleic acid markers for cell health or cell pathology. These techniques include Western blotting, ELISA, immune-histochemical techniques, and nucleic acid analysis, i.e northern blotting, RT-PCR (reverse transcriptase polymerase chain reaction) (See Satchell, S. et al., 2016. “Conditionally immortalized human glomerular endothelial cells expressing fenestrations in response to VEGF,” Kidney International, 69:1633-1640; and Singh, A. et al., 2007. “Glomerular endothelial glycocalyx constitutes a barrier to protein permeability,” J Am Soc Nephrol, 18:2885-2893).

### XI. Molecular Biomarkers for Assessing Model Systems

Biomarkers are a diverse group of molecular moieties that can identify cellular health or cell pathophysiology, or simply detect whether various cell types are present or absent. Biomarkers for endothelial cell and podocyte cell physiology are known, and find use with the invention. These biomarkers include markers indicative of mature differentiated cells types, which finds use with the invention in the determination of the presence or absence of the relevant cell types in the bioreactor cultures.

Relevant kidney biomarkers find a variety of uses with the invention, for example, in the study of health, aberrant physiology or pathology (i.e., disease) of the cells maintained in any of the cell co-culture systems of the invention, i.e., cell co-culture systems for the growth and maintenance of glomerulus-derived vascular endothelial cells and podocyte cells.

Biomarkers can be protein biomarkers or nucleic acid biomarkers. In the case of nucleic acid, biomarkers, as used herein, the biomarker will commonly be expressed gene markers, i.e., mRNA. A wide variety of methodologies for the assessment of molecular markers are well known. For example, a protein marker can be detected by any suitable immunoassay, such as ELISA or Western blotting. Markers that are mRNA markers can be detected, for example, by collecting a cell sample and performing quantitative or non-quantitative RT-PCR. High-throughput screening for many different expressed RNA markers can be done using a suitable array or chip nucleic acid probe analysis. Urinary biomarker levels can be estimated from the permeate, which is analogous to the glomerular filtrate, after normalization for the number of fibers, appropriate scaling and accounting for the reabsorption that takes place. Alternatively, permeate levels can be compared directly between health and disease models. Serum levels of the biomarkers can be estimated from the retentate. Retentate levels can be compared directly between health and disease models. Alternatively, after normalization for the number of fibers and appropriate scaling, the retentate serum levels can be compared to clinical values.

In some embodiments, the molecular markers used with the invention are circulating markers, that is to say, they are secreted into the culture medium, and can be found in either the culture retentate or in the culture permeate. In other embodiments, the molecular markers remain affixed to or within the relevant cells. In order to analyze those type of non-circulating markers, cells are harvested from the bioreactor and samples are prepared for either a protein analysis (e.g., ELISA or Western blotting) or a nucleic acid analysis (e.g., RT-PCR).

Commonly used biomarkers for glomerular endothelial cells are von Willebrand factor (vWF), platelet endothelial cell adhesion molecule 1 (PECAM1), CD31, vascular endothelial (VE)-cadherin (CD144) and growth factor receptors vascular endothelial growth factor receptor 2 (VEGFR2) and Tie2. See Satchell et al., “Conditionally immortalized human glomerular endothelial cells expressing fenestrations in response to VEGF,” Kidney International, 69: 1633-1640 (2006).

Biomarkers can be used for different purposes. For example, the presence or absence of a biomarker at any onetime point can be indicative of cell health, normal physiology and normal cell differentiation, or conversely, can indicate the presence of disease, toxicity or pathophysiology. Thus, when that biomarker is detected, or detected above or below a certain expression threshold, normal cell physiology or cell pathophysiology is indicated. Similarly, an upward trend or downward trend in the concentration of a biomarker over a period of time can indicate improvement in cell functioning or worsening of a pathophysiogical status.

Increased urinary levels of ACTN4 mRNA (encodes for α-ACTN4, a urinary biomarker for podocytopathy) and increased urinary levels of CD-2 associated protein (CD2AP, a urinary biomarker for podocytopathy) indicates the presence and monitors the progression of diabetic nephropathy (DN). The presence and disease progression of DN is also revealed by increased serum levels of tumor necrosis factor-α (TNF-α; a cytokine). Increased urinary levels of CD80 (B7-1) mRNA (encoding for CD80 protein), indicates the presence of focal segmental glomerulosclerosis (FSGS), while Interleukin (IL)-1B, intracellular adhesion molecule 1, vascular cell adhesion molecule −1 and vascular endothelial growth factor (VEGF) are upregulated in diabetes milletus (DM). Some biomarkers, such as nephrin mRNA, when present in increased levels in the urine, can indicate the presence, progression and recovery of several diseases, including DN, FSGS, IgA nephropathy (IgAN), nephrotic syndrome (NS) and pre-eclampsia (PE). Sekulic, S. and M. Sekulic, 2013. “A compendium of urinary biomarkers indicative of glomerular podocytopathy,” Pathology Research International, Volume 2013, Article ID 782395, Hindawi Publishing Corporation; Taneda et al., 2015. “Podocyte and endothelial injury in focal segmental glomerulosclerosis: an ultrastructural analysis,” Virchows Arch., 467(4): 449-458.

Additional information regarding the use molecular markers in the assessment of renal (glomerular) health and disease is known in the art. Molecular markers of any type can find use with the invention, and are not limited to any markers specifically recited herein. For additional discussion of the use of molecular markers finding use with the invention, see for example but not limited to: Chittiprol et al., 2011. “Marker expression, behaviors, and responses vary in different lines of conditionally immortalized cultured podocytes,” Am J Physiol Renal Physiol., 301(3): F660-671; Khan and Pandey, 2014. “Role of kidney biomarkers of chronic kidney disease: an update,” Soudi J. Biol. Sci., 21(4): 294-299; Krtil et al., 2007. “Culture methods of glomerular podocytes,” Kidney Blood Press Res., 30: 162-174; Kumar, P. A., et al. 2014. “Molecular and cellular events mediating glomerular podocyte dysfunction and depletion in diabetes mellitus.” Frontiers in Endocrinology. 5: 1-10. Navarro-Gonzalez and Morao-Fernandez, 2008. “The role of inflammatory cytokines in diabetic nephropathy,” J. Am. Soc. Nephrol., 19: 433-422; Sekulic, S. and M. Sekulic, 2013. “A compendium of urinary biomarkers indicative of glomerular podocytopathy,” Pathology Research International, Volume 2013, Article ID 782395, Hindawi Publishing Corporation; Sun et al., 2013. “Glomerular endothelial cell injury and damage precedes that of podocytes in adriamycin-induced nephropathy,” PLoS ONE 8(1): e55027; and Taneda et al., 2015. “Podocyte and endothelial injury in focal segmental glomerulosclerosis: an ultrastructural analysis,” Virchows Arch., 467(4): 449-458.

### XII. Methods for Assessing Drug Toxicity and Predicting Drug Efficacy in the Treatment of Renal (Glomerular) Disease

The glomerular cell co-culture model systems of the invention are well suited to study of effects of any particular treatment on the endothelial cell and podocyte cell co-cultures of the invention. In some embodiments, these effects are drug effects following exposure of the glomerular cell co-culture model system to a drug of interest. Observing the effects of a treatment comprises observing changes in a functional measure or physiological marker in response to a treatment. Any of a variety of suitable physiological parameters can be monitored as a measure of cell health, or conversely, as a measure of cell toxicity or any other negative impact on normal cell function as a result of the treatment.

Normal, healthy cells in a hollow fiber bioreactor of the invention can be treated in order to assess the toxic (or non-toxic) results of a particular treatment on the cultured cells. Employing the model culture system of the invention for this purpose has use in predicting the in vivo impact, i.e., the functional measure response or the cell physiological response, of administering a treatment to a person, such as a drug treatment, and in particular, is predictive as to the effects of administering that drug on glomerular cell co-culture model system function.

Conversely, diseased or impaired cells that are cultured in a bioreactor of the invention can be used to model the potential impact in treating a person for a renal (glomerular) disease. For example, if a cell culture of the invention is maintained using a configuration or diseased cells that result in abnormal cell function or abnormal glomerular system function, it can be observed whether a treatment such as a drug exposure can restore or improve health or proper function of those cells or the glomerular system function.

The invention provides methods well suited for assessing the effects of any particular treatment of interest on the co-cultured cells in the apparatus of the invention, namely glomerulus-derived vascular endothelial cells and podocyte cells. As used herein, the terms “treatment” or “treatment condition” or “treatment parameter” or similar and equivalent terms are broadly defined to mean any change in the cell co-culture environment. Treatments can include drug treatments, although other types of treatments are contemplated for use in methods of the invention, for example, changes in engineering variables or culture conditions such as elevated pressures in the fiber luminal space and nutritional state of the cell culture medium (e.g., elevated glucose mimicking in vivo hyperglycemia). As used herein, the expressions “effects” or “physiological effects” or “functional measures” or “physiological response” to any given treatment can include functional parameters such as any classical measure of renal physiological function that can be modeled in the co-culture apparatus of the invention, for example, by quantitating a permeate flow rate (analogous to a glomerular filtration rate), red blood cell count/mL of permeate or a sieving coefficient, such as an albumin sieving coefficient or a total protein sieving coefficient, which can be calculated from the permeate and retentate of the apparatus of the present invention. In other aspects, the “effects” of a treatment can be molecular characterizations, such as the absence, presence, induction or reduction in any relevant molecular marker that finds use in the assessment of, for example, cell health, cell growth, cell differentiation, cell toxicity or cell pathology.

Essentially, observing the response of the cells to a treatment comprises first observing or measuring a cell physiological trait or system functional measure prior to the treatment, followed by treatment of the cells in the bioreactor culture, then again observing or measuring that same cell physiological trait or system functional measure at a time point(s) after the treatment. Additionally, responses due to treatment can be compared to those of controls.

Subjecting a glomerular cell co-culture model system to a treatment comprises of either (I) altering a culture parameter or altering an equipment parameter to control or vary a biological engineering variable in the partitioned cell co-culture system, or (II) delivering a drug or drug candidate to the partitioned cell co-culture system.

Subjecting the cell co-culture glomerular model system to a drug treatment comprises delivering a drug or drug candidate, in some embodiments pre-treated with e.g. liver tissue homogenate S9 subcellular fraction, in order to pre-metabolize the drug to mimic the in vivo bioavailable form of the drugt. See DiPiro, J. T. et. al., 2014. “Pharmacotherapy: a pathophysiological approach.” McGraw-Hill, New York.

The glomerular cell co-culture model system is exposed to a control condition(s) for each treatment to assess the effects of the treatment versus another confounding factor, and to compare effects of treatment to a baseline response.

For example, a candidate drug can be tested on healthy cells in the bioreactor of the invention as a predictor for potential impact on kidney function in a healthy person. A bioreactor of the invention can be inoculated and cultured using healthy glomerulus-derived microvascular endothelial cells and with healthy podocyte cells. Any suitable physiological trait is first measured, such as any of the traditional measures of kidney function including the filtration rate through the fiber walls, i.e., through the fiber walls in conjunction with the attached cell layers and any natural, naturally secreted or artificially applied coatings or cellular-deposited basement membrane material. The rate of flow through the fiber walls is analogous to the in vivo glomerular filtration rate, in particular, if whole blood is used in the experimental system. Other system parameters or traits that can be assessed include total protein concentration in the permeate flow, albumin polypeptide concentration in the permeate flow (if whole blood is circulated in the culture system at the time of the analysis), and red blood cell concentration (red blood cells/mL of permeate) in the permeate flow (when whole blood is used in the test system).

Alternatively, one or more molecular markers indicating cell health or disease can be analyzed in the model system, for example, in the culture permeate that has passed though the bioreactor fiber walls, which is analogous to a glomerular filtrate that is produced in vivo. Alternatively, or in addition, one or more molecular markers can be assessed in the culture medium retentate produced by the model system, that is to say, the portion of the culture medium that does not pass through the fiber walls and passes through the full length of the hollow fiber capillaries. This model system retentate material is analogous to the efferent blood supply that exits the glomerular capillary bundle. Markers such as CD31, synaptopodin, cystatin C or WT1 can be assessed. The simple presence or absence of a protein or mRNA marker may be sufficient, or the marker can be assayed quantitatively.

### Application to the Hollow Fiber Bioreactor Model System:

Cells in the bioreactor are originally seeded and grown using traditional defined cell culture media. To conduct a study to observe the effects of exposure of a drug on the function of this bioreactor model that mimics the glomerular filtration function of generating a glomerular filtrate, a media change to whole blood can be made.

Next, the cells in the culture are exposed to a treatment, such as a drug treatment. See FIG. 9 that provides an example of an apparatus optimized for this type of testing. In some preferred embodiments for drug testing protocols, whole blood is used as the culture medium, although the systems of the invention are not limited to the use of whole blood as a culture medium. As described herein, FIG. 9 is optimized for drug testing protocols. However, drug testing protocols can be conducted in any apparatus of the invention, and are not limited to use of the apparatus depicted in FIG. 9.

As shown in FIG. 9, the drug will be administered to the system contained in the culture medium, for example, in whole blood. In some embodiments, a drug solution of appropriate concentration is pre-treated, for example, with a liver tissue homogenate S9 subcellular fraction, in order to pre-metabolize the drug to mimic the in vivo bioavailable form of the drug, as known in the art.

Whole blood supplemented with a first drug is pumped from a reservoir 950 either alone or in combination with whole blood containing a second drug, which is pumped from a second reservoir 990 into the feed reservoir 940 from lines 952 and 992, respectively. Reservoir 962 can be filled with whole blood without drugs (diluent), which is used to dilute the drug concentration in the feed reservoir 940. The purpose of this configuration is to allow fine tuning of drug concentration in the central feed reservoir 940 that supplies the bioreactor cartridge 900, or to mimic elimination kinetics. The drug concentration (i.e., drug dosage) in the feed reservoir 940 is finely controlled by the delivery of proper proportions of both drug-containing medium from the e.g. drug reservoir 950 and drug-free culture medium from the diluent reservoir 960.

Testing is initiated when whole blood with or without drug(s) is pumped in a tangential directional flow from the feed reservoir to the HFBR 900 via feed line 942. The material that travels the full length off the fiber lumen and passes through to the retentate line 982 and is e.g. collected in a retentate reservoir 980. Thus, the retentate does not get recycled back to the cartridge, so that especially blood containing drug does not get recycled back into the model system. The permeate passes through the semipermeable membrane under positive pressure and is collected in a permeate reservoir 970 after passing through a permeate flow line 972. If present, drug(s) will be allowed to be filtered out across the fiber wall in the permeate, analogous to the function of the glomerulus and Bowman's capsule.

The process that controls drug concentration (i.e., drug dosage) in the co-culture system can be controlled manually by an operator using any arrangement of valves, pumps, and pumping rates through the relevant supply lines. Alternatively, the process that regulates drug concentration (i.e., dosage) or any other parameter of the co-culture system can be controlled electronically by a programmable processor unit. The processor unit can be programmed and instructed to monitor and control valve positions, pump activation or deactivation, and/or pumping rates through the relevant supply lines in order to achieve a desired result, for example a desired drug concentration in the feed reservoir 940. In some embodiments, the apparatus is programmed to deliver fixed concentration of drug to the cell co-culture during the testing interval, or alternatively or in combination, can also be programmed to increase the drug concentration or decrease the drug concentration at a fixed rate over a designated time interval. In some embodiments, the systems of the invention further comprise sensors that can measure drug concentration in real time anywhere along the culture medium circuit, deliver that feedback metric to the processor unit, and where in response the processor unit control culture medium delivery in order to fine tune the concentration of the drug under study.

Experiments to determine the effects of particular drugs can be run with one or more controls i.e., i) whole blood without drug(s) and without excipient and ii) whole blood with excipient and without drug(s). Experimental runs using iii) whole blood and excipient with a first drug, a second run using iv) whole blood and excipient with a second drug and a third run using v) whole blood and excipient with both the first and second drug may be conducted for comparison to each other and the controls to see the effects of changes in the glomerular model system function and cell physiology prior to treatment versus at a time after treatment.

Experiments for determining the effects on glomerular model system function and cell physiology of different drug dosages can be conducted by comparing experimental runs (using whole blood) employing different concentrations of drug to controls consisting of i) whole blood without drug and with excipient, as well as ii) whole blood without drug and without excipient.

Experiments for determining the effects of drug dosage intervals on the glomerular model system function and cell physiology can be conducted with controls, i.e. i) control 1—whole blood without excipient and without drug; ii) control e.g. 2a to 2e—whole blood with excipient and without drug for each dose interval to be tested; iv) control 3—constant flat line delivery of whole blood with excipient and with particular drug dose tested; v) experiments e.g. a-e using different dosage intervals for particular drug dose tested.

Experiments for determining the effects of concentration gradients (i.e. change in concentration over a time interval) of a drug on the glomerular model system function and cell physiology (including mass transfer of drug) can be run with controls, i.e. i) control 1—whole blood without excipient and without drug; ii) control e.g. 2a to 2e—whole blood with excipient and without drug for each gradient to be tested; iii) control e.g. 3a to 3h—constant flat line of drug in excipient for representative concentrations used in the particular gradient tested for the time interval of that gradient; iv) experiments e.g. 4a to 4e run with excipient and drug for different concentration gradients (i.e. change in concentration over particular time interval).

A sufficient volume of the test blood containing the drug will be utilized to allowed for the required time of exposure to the drug concentration of interest, i.e.: volume of test blood required=(feed flow rate×time of exposure)−(permeate flow rate×time of exposure).

Prior to treatment and at a time point(s) after initiating treatment, the originally tested functional measure or cell physiological trait is analyzed again. Changes in that functional measure or cell physiological trait or marker after the treatment can indicate continued cell health or healthy glomerular model system function, or conversely, deterioration in cell health or deterioration in glomerular model system function.

In the scenario where the bioreactor of the invention is established using culture conditions and cells that mimic a healthy glomerular state, and using whole blood or a blood fraction derived from a healthy animal or a healthy human, if the cell physiological trait or functional measure is indicative of cell health and healthy renal (glomerular) model system function, then observing no change in that physiological trait or functional measure following drug treatment is a sign of continued cell health and healthy glomerular model system function, respectively. This is predictive of there being no impact on in vivo kidney function if that drug was to be administered to a healthy person.

In the scenario where the bioreactor of the invention is established using culture conditions and cells that mimic a healthy glomerular state, and using whole blood or a blood fraction derived from a healthy animal or a healthy human, and if the cell physiological trait or functional measure is a marker or measure for cell toxicity and impaired renal (glomerular) model system function, i.e. reduced filtering capacity or poor filtering integrity, then an increase (or first appearance) of that marker or functional measure after drug treatment is indicative of cell toxicity or impaired renal glomerular model system function, respectively. This may be predictive for toxic effects or impairment of kidney function if that drug was to be administered to a healthy person.

Conversely, a bioreactor of the invention can be configured to model a disease state or an aberrant renal (glomerular) system function. This is done, for example, by using cultured cells derived from diseased tissue, using cells that phenotypically mimic some aspect of renal (glomerular) disease, using cells containing one or more genetic anomaly known to be involved in regulating renal (glomerular) function, using modified operating conditions such as higher pressures in the fiber lumen, using modified equipment such as increased porosity of the hollow fiber walls, and/or using blood or a blood fraction derived from a diseased animal or a diseased human patient.

When the bioreactor is established to model renal (glomerular) pathology, then an increase or first appearance following drug treatment of a relevant functional measure or cell physiological trait or biomarker that is indicative of healthy glomerular model system function or cell health, respectively, indicates improved glomerular model system function or improved cell health, respectively. This may be predictive of an improvement in kidney function in a patient if that drug was to be administered to a patient having an analogous kidney disease.

Similarly, when the bioreactor is established to model renal pathology, then a decrease of a functional measure or loss of a cell physiological trait or marker indicative of disease, aberrant renal (glomerular) model system function or cell pathology following the drug treatment, indicates improvement in glomerular model system function and improved cell health, respectively. This may be predictive of an improvement in kidney function following drug treatment in a diseased patient if that drug was to be administered to a patient having analogous kidney disease.

### XIII. Incorporation of Conditioned Cell Culture Medium or Conditioned Whole Blood for Benefits of Additional Cell Types.

An additional cell type that is present in the glomerulus is the mesangial cell. In vivo, mesangial cells are closely associated with endothelial cells and podocytes and contribute to the physical architecture of the glomerulus and the maintenance of capillary loops. They also have a role in filtration regulation via control of the glomerular blood volume and the filtration surface area (Stockand, J. and S. Sansom. 1998. “Glomerular mesangial cells: electrophysiology and regulation of contraction.” Physiological Reviews, 78 (3): 723-744).

The benefits of mesangial cell secreted substances in the bioreactors of the invention can be achieved by using conditioned cell culture media or conditioned whole blood, in part or in whole, in the bioreactor, where that conditioned media has been previously exposed to mesangial cell culture and as a result, contains substances that are secreted by the mesangial cells. Alternatively, the cell culture medium or whole blood used in the bioreactor can be supplemented in part by a fractional volume of mesangial cell-conditioned cell culture medium or mesangial cell-conditioned whole blood medium. This will enable the system to benefit from the growth factors and cytokines (e.g., platelet-derived growth factor (PDGF), macrophage colony-stimulating factor (CSF1), transforming growth (TGF-β) and Interleukin-1 (IL-1)) produced by mesangial cells (see Schlondorff, D. 1996. “Roles of the mesangium in glomerular function,” Kidney International, 49: 1583-1585). According to the literature (see Schlondorff, D. 1996. “Roles of the mesangium in glomerular function,” Kidney International, 49: 1583-1585), PDGF, for example, has been directly shown to be involved in the generation of glomerular capillary loops as well the maintenance of these vessels.

Additionally, plasminogen activator and inhibitor and the growth modifying agents (e.g., prostaglandins, platelet activating factor, or PAF), and vasoactive media (e.g., nitric oxide) can be present in the mesangial conditioned culture system.

Since mesangial cells are not present themselves in the HFBR model, vasodilation and vasoconstriction can be represented by equipment size change (hollow fiber/tubing size increase or decrease) and efferent arteriole pressure can be altered by altering retentate control valve setting. The advantage of this model in studying the effects of mesangial contraction (which causes glomerular capillary contraction, see Ghayur, M., et al. 2008. “Contractility of the renal glomerulus and mesangial cells: lingering doubts and strategies for the future.” Med Hypotheses Res. 4 (1): 1-9.) is that the hollow fiber (analogous to the glomerular capillary) size can be reduced without affecting the feed tubing (analogous to the afferent arteriole), thus uncoupling the effects of the glomerular capillary analogue contraction from the effects of the afferent arteriole analogue contraction (which impacts the renal blood flow rate). This is not possible using other investigative tools that use agonists that cause contraction of both the afferent arteriole and the glomerular capillary.

### XIV. Methods for the Study of Proximal Tubule Function

The glomerular filtrate in vivo is further processed in the renal proximal tubules, where metabolic waste products in the glomerular filtrate are concentrated in the filtrate fluid stream and where various components of the glomerular filtrate are reabsorbed back into the circulatory system (Truskey, G. A. et al. 2009. “Transport Phenomena in Biological Systems”. Pearson Education, New Jersey). The invention finds use for the generation of a model glomerular filtrate that can be used in studies that examine proximal tubule function.

The cell co-culture model system permeate that is generated by the methods and apparatus of the invention find use in the study of renal proximal tubule cells, i.e., proximal tubule epithelial cells. The proximal tubule cells are exposed, in part or in whole, to the permeate (analogous to the glomerular filtrate) generated by methods of the invention when whole blood is used as the culture medium. Thus, a fluid stream is provided that more closely mimics the in vivo environment of the proximal tubule cells, compared to other types of proximal tubule cell culture systems, in order to study the physiology of proximal tubule cells. Additionally, the invention also provides a permeate stream, analogous to the glomerular filtrate, that has been generated in response to the treatment of the glomerular co-culture model systems with appropriate concentrations of a pharmacological agent using whole blood as the medium. The filtrate produced is then used to treat proximal tubule cells and observe toxicity in those cells. For example, the model glomerular filtrate of the invention can be exposed to porcine LLC-PK1 (pig kidney) cells or marsupial OK (opossum kidney) cells, which are derived from proximal tubule epithelial cells and commonly used in the study of kidney disease.

### EXAMPLES

The following examples are offered to illustrate, but not limit, the claimed invention. It is understood that various modifications of minor nature or substitutions with substantially similar reagents or components will be recognizable to persons or ordinary skill in the art, and these modifications or substitutions are encompassed within the spirit and purview of this disclosure and within the scope of the invention.

### Example 1

### Hollow Fiber Bioreactor Models for Renal (Glomerular) Health and Renal (Glomerular) Disease Model

This example provides a description of a hollow fiber bioreactor cell co-culture models of a glomerulus used to simulate renal (glomerular) model systems in both health and diseased conditions finding use with the invention.

Cells are co-cultured in a e.g. FiberCell® Systems (Frederick, Md.) PS+ module cartridge (small cartridge, Catalog No. 4300-C2025). The hollow fibers in this cartridge are made from polyvinylidene fluoride (PDVF), are 10 cm in length, have an inner diameter of 700 μm and outer diameter of 1300 μm. The fiber walls have a pore size of 0.1 μm as rated by the manufacturer. The cumulative surface area of all the fibers (20 fibers) in the system is 75 cm² and the extra-capillary space (ECS) volume is 2.5 mL. The FiberCell® Systems (Frederick, Md.) PS+ fibers have a surface chemistry that facilitates the attachment of extra-cellular matrix proteins, antibodies, cytokines or other advantageous protein or non-protein components to the surface of the fiber, and in particular, for attaching matrices that promote the attachment of endothelial cells to the interior wall of the fiber. These fibers have the further benefit of enduring most types of microscopy, including immune-histochemical techniques, thereby permitting morphological analysis and immunological characterization of the cultured cells (see FiberCell® Systems cartridge catalogue C2025).

For the case of a healthy glomerulus HFBR model, the 0.1 μm MWCO is altered when either the hollow fiber material is either customized or altered to minimally achieve effective exclusion (90% exclusion) of a benchmark protein. In this case, the benchmark protein is albumin protein (˜65 kDA). Alternatively, different support material having a greater filtering capacity (lower MWCO) is incorporated into the bioreactor cartridge fibers at the time of manufacture.

If necessary, the sieving properties of the porous fiber wall are modulated by the addition of one or more coating onto the fiber wall which will hinder the sieving capacity of the fiber wall. For example, a coating of an extracellular matrix such as Matrigel® extra-cellular matrix material (Corning® Life Sciences) or natural GBM proteins can hinder the filtering capacity of the fiber walls. A plurality of coatings of different materials can be used to modify the filtering capacity of the fibers.

Following the application of any extra-cellular matrix or glomerular basement membrane components to the fiber walls, the cells are inoculated into the bioreactor cartridge. A model system for normal (i.e., healthy) renal function is created by the inoculation of glomerular endothelial cells onto the luminal (i.e., inner) surface of the fiber. In preferred embodiments, healthy, human glomerular endothelial cells are used, where those cells are not associated with any known renal (glomerular) pathology (i.e., those cells were isolated from or derived from healthy patients and/or do not contain any known genetic defects that may contribute to renal (glomerular) pathology). Similarly, healthy human podocyte cells can be used to inoculate the outer surface of the fiber in the extra-capillary space of the bioreactor (i.e., podocyte cells that were isolated from or derived from healthy patients and/or do not contain any known genetic defects that may contribute to renal pathology). This model system configuration mimics normal function of the in vivo glomerular filtration apparatus.

In other embodiments, it is the objective to mimic the aberrant production of glomerular filtrate in various renal disease states characterized by loss of filtering capacity in the glomerular filtration apparatus. In that case, grades of severity of pathology of glomerular filtration can be modeled by using hollow fibers with larger molecular weight cut-off values that do not restrict the passage of albumin across the hollow fiber walls, or ineffectively exclude, to different desired degrees, the reference protein albumin. Here, one could use diseased i.e. human endothelial cells on the luminal hollow fiber surface, and diseased podocyte cells on the outer fiber surface in the extra capillary space.

Modeling of renal (glomerular) disease can also be accomplished, following the application of any extra-cellular matrix or glomerular basement membrane components to the fiber walls, by inoculating the cells into the bioreactor cartridge by inoculating the partitioned bioreactor that are known to be associated with disease states. For example, using cells that have been isolated from patients with renal (glomerular) disease and/or using cells that are genetically engineered to contain genetic defects that are known to contribute to renal (glomerular) disease or renal (glomerular) pathology. Diseased glomerular endothelial cells can be inoculated into the luminal (i.e. inner) surface of the hollow fiber(s) and/or diseased podocyte cells can be inoculated on the outer surface of the hollow fiber(s) in the extra capillary space. This configuration mimics in vivo the diseased state of the glomerular filtration apparatus.

### Example 2

### Hollow Fiber Bioreactor (HFBR) Model System No. 2—Cell Culture Establishment

This example describes the inoculation and co-culture maintenance of a hollow fiber bioreactor of the invention.

Glomerular endothelial cells are cultured in a suitable culture media with VEGF (5 ng/mL) for one week in order to avoid subsequent detachment of podocyte cells in the glomerular co-culture with podocyte cells in the HFBR. See Satchell et al., 2006. “Conditionally immortalized human glomerular endothelial cells expressing fenestrations in response to VEGF,” Kidney International, 69: 1633-1640.

The glomerular endothelial cells are seeded onto the inner lumen of the hollow fibers in the bioreactor. After all ports are closed, place the HFBR in the incubator for one hour, rotating the HFBR 180 degrees at the halfway point without any flow so that the endothelial cells can attach (see FiberCell Systems. 2015b. “User Manual). Following introduction of the cells into the luminal space of the hollow fibers, the feed port and retentate port that would otherwise service the luminal space are closed. At this time, media is circulated through the outer side ports, i.e., the ports that access the extra-capillary space on the outside of the hollow fibers. The cells are maintained in this state over several hours or days to permit attachment of cells.

For the next step, podocyte cells are seeded into the extra-capillary space and the side ports accessing the extra-capillary space are closed. The media flow is then switched and takes place from the feed port through the retentate port through the hollow fiber luminal spaces.

The FiberCell® Systems C2025 cartridge is coupled to tubing, a medium reservoir and pumping apparatus also provided by the same manufacturer, for example, the FiberCell” Systems Duet Pump System (Catalog No. P3202). A microprocessor controls the pumping system to produce a consistent and defined level of shear stress over the glomerular-derived endothelial cells which can be cultured on the interior surface of the hollow fibers in the luminal space. Shear stresses of 0.5 dynes/cm² to 25 dynes/cm² can be generated.

Growth conditions and procedures for culture establishment generally follow protocols known in the art, for example, according to the manufacturer's recommendations. The cell co-culture can be maintained in batch mode using recirculated culture medium. In the batch mode configuration, there is no segregated permeate flow stream.

One example of a co-culture system configured for batch-mode operation (with no permeate flow) is shown in FIG. 5. The culture medium resides in a central culture medium feed reservoir 540. The medium is channeled through a feed line 560 driven by an in-line feed pump 546. The culture medium travels through gas-exchange tubing in an in-line oxygenation apparatus 550 for oxygenation of the culture medium and removal of dissolved carbon dioxide, into the lumen of the hollow fibers 508 and back to the central feed reservoir 540.

Growth conditions and procedures for culture establishment generally follow the descriptions for optimal culture of the particular cells deployed in the co-culture, for example, as described in the scientific literature or provided by the distributor of the cells. The glucose consumption rate is proportional to the cell growth rate, cellular metabolic activity and cell number, and is approximately in the range of 1.0-1.5 grams/day, for example, if using FiberCell® Systems cartridge catalog No. 2008 to estimate glucose consumption (see FiberCell Systems. 2015b. “User Manual). The glucose levels are monitored. When the glucose level drops below a desired threshold (e.g., less than 50% of the starting glucose concentration in fresh culture medium), the media bottle is replaced with a new media bottle. Gas exchange occurs via a gas permeable silicon tube in the flow path and is controlled, along with the temperature, by placing the system in a CO₂ incubator. The co-culture is allowed to establish and is maintained until the commencement of any experimental stage.

### Example 3

### Assays for Glucose and Albumin

This example describes testing protocols for glucose in a defined cell culture media during cell culture establishment. In the scenario of a batch mode (retentate recycle) system (FIG. 5), the media samples are obtained at the time of each medium replacement and analyzed, for example, for glucose.

Glucose Assay.

A kit provided by Sigma-Aldridge, Inc.® (catalog# GAGO20-1KT) can be used to determine glucose levels, more specifically, the change in glucose level from the previous day. The reagent employed causes, over a 15-minute incubation, the conversion of glucose to glucose-6-phosphate through a phosphorylation reaction, followed by the oxidation of the latter in the presence of NAD (nicotine adenine dinucleotide) to 6-phosphogluconate. The oxidation reaction results in an equal number of moles of NAD being reduced to NADH, the latter of which increases the absorbance of the sample at a 340 nm wavelength as measured by a spectrophotometer. This signal is directly proportional to the glucose concentration (Sigma-Aldridge, 2015).

Albumin Assay.

In some embodiments, the media can be whole blood that is circulated through the co-culture apparatus after the cell populations have been established using defined culture medium. At this time, i.e. during glomerular cell co-culture model system operation (with permeate flow stream), as shown in FIGS. 6-9, albumin can be assayed as a glomerular system functional measure. Either the permeate flow stream (for the permeate concentration) or both the retentate flow stream and permeate flow stream (for the sieving coefficient) can be subjected to testing. The albumin assay is conducted for healthy and diseased glomerular models as well as after drug treatment or drug toxicity experiments.

An albumin assay kit is available that allows albumin to be measured with little interference from lipids and other proteins and with no pre-treatment of the sample. See Sigma-Aldrich, Inc.® Bromocresol Green (BCG) albumin assay kit (catalog# MAK124). The BCG reacts with the albumin to form a stable colored complex after a 5-minute incubation. The intensity of the color can be measured in a spectrophotometer at a 620 nm wave length, providing a signal that is directly proportional to the concentration of the albumin (Sigma-Aldrich, 2015).

### Example 4

### Hydrostatic and Oncotic Pressure Testing

This example describes the hydrostatic and oncotic pressure testing that can be performed on the hollow fiber bioreactor systems of the invention. Depending on the glomerular HFBR chosen, the glomerular capillary hydrostatic pressure (average pressure of bioreactor feed pressure and retentate pressure), and the Bowman's capsule hydrostatic pressure is mimicked, or at least, the feed and retentate pressure can be manipulated to obtain a representative net filtration pressure (NFP).

The values of the hydrostatic and oncotic pressure of the afferent and efferent arterioles, as well as in the Bowman Capsule are detailed below. The hydrostatic pressure of the afferent and the efferent arterioles result from the renal arterial and venous pressures as well as the control exerted by the vasomotor center of the brain on the afferent and efferent arterioles, while the oncotic pressure is a function of plasma protein concentration (See Truskey, G. A. et al. 2009. “Transport Phenomena in Biological Systems”. Pearson Education, New Jersey).

One of the measures of the functionality of the kidney (including deterioration or improvement upon treatment) is the NFP (net filtration pressure), glomerular filtration rate (GFR) and the various permeate concentration measures/sieving coefficients (which measure the extent to which protein, albumin or red blood cells leak across the membrane).

These conditions, or at least the resulting net filtration pressure (NFP), can be reproduced using the tangential flow filtration (TFF) glomerular model in the culture systems of the presently described invention. In the glomerulus filtration model, the feed flow rate and the retentate control valve can be used to control the feed pressure (P_{F}) and retentate pressure (P_{R}), respectively. The net filtration pressure (for filtration from the glomerulus to the Bowman's capsule in the production of glomerular filtrate, and by analogy, from the bioreactor feed line to the ECS permeate) can be described by net filtration pressure (NFP):

NFP=(P_{gc}−P_{bc})]−(π_{gc}−π_{bc})]; where

P_{gc}=glomerular capillary hydrostatic pressure (mm Hg)=average pressure of bioreactor feed pressure and retentate pressure

P_{bc}=Bowman's Capsule hydrostatic pressure (mm Hg)=permeate pressure

π_{gc}=glomerular capillary oncotic pressure (mm Hg)=avg. oncotic pressure of feed and retentate

π_{bc}=Bowman's Capsule oncotic pressure (mm Hg)=oncotic pressure of exterior surface of the hollow fiber

In humans, this typically is:

$$\begin{matrix}NFP = ( {P}_{gc} - {P}_{bc} ) ] - ( {π}_{gc} - {π}_{bc} ) \\ = ( 50 - 17 ) - ( 25 - 0 ) \\ = 8 mm Hg\end{matrix}$$

where:
P_{gc}=glomerular capillary hydrostatic pressure (mm Hg)˜50 mm Hg
P_{bc}=Bowman's Capsule hydrostatic pressure (mm Hg)˜17 mm Hg
π_{gc}=glomerular capillary oncotic pressure (mm Hg)˜25 mm Hg
π_{bc}=Bowman's Capsule oncotic pressure (mm Hg)˜0 mm Hg

The size (diameter) of the tubing/hollow fiber can be manipulated (in addition to constricting the retentate tubing with a control valve), since one of the ways that the vasomotor region of the brain controls blood pressure is via the diameter of the blood vessels. Thus, experimental NFP values in the bioreactors of the invention can be adjusted to mimic in vivo NFP values, thereby providing a modeling variable that finds use in the study of normal and disease glomerular filtration mechanisms. In addition, the impact of drugs (e.g., vasoconstrictors and vasodilators) can be mimicked by altering equipment parameters, i.e., the feed tubing size, hollow fiber size and altering the retentate control valve setting.

### REFERENCES

Abrahamson, D. 2009. “Development of kidney glomerular endothelial cells and their role in basement membrane assembly,” Orgonogenesis, 5 (1): 275-287.
Abrahamson, D. 2012. “Role of the Podocyte (and Glomerular Endothelium) in Building the GBM,” Semin Nephrol., 32 (4): 342-349.
Aisimo Corporation LTD, 2013. “Table relating kilodaltons to microns”.
American Society for Hospital Pharmacists.
Ballerman, B.; Ott, M. 1995. “Adhesion and differentiation of endothelial cells by exposure to chronic shear stress: a vascular graft model.” Blood Purif. 13: 125-34.
Bruggeman, L. et al. 2012. “A cell culture system for the structure and hydrogel properties of basement membranes; Application to capillary walls.” Cell Mol Bioeng. 5(2): 194-204.
Byron, A. 2013. “Glomerular cell cross-talk influences composition and assembly of extracellular matrix,” J Am Soc Nephrol., 25(5): 953-966.
Cadwell, J. 2004. “New developments in hollow fiber cell culture.” American Biotechnology Laboratory
Cadwell, J. Jun. 12, 2012 “The Hollow Fiber Bioreactor and Cell Co-Cultivation,” American Laboratory article
Cadwell, J. 2012. “The hollow fiber infection model for antimicrobial pharmacodynamics and pharmacokinetics.” Advances in Pharmacoepidemiology and Drug Safety. 51: 007.
Caret, R. L. et., 1995. “Foundations of inorganic, organic, and biological chemistry.” Wm. C. Brown Publishers, Boston.
Chittiprol, S. et al. 2011. “Marker expression, behaviors, and responses vary in different lines of conditionally immortalized cultured podocytes,” Am J Physiol Renal Physiol. 301 (3): F660-671.
Chuang et al., 2013. “Capturing the in vivo molecular signature of the podocyte,” Kidney International, 83(6): 986-988.
Dane, M. J. 2013. “Glomerular endothelial surface layer acts as a barrier against albumin filtration,” Am J of Pathology, 182 (5): 1532-1540.
Dergosits, M. 2016. Private communication—pulsatile pump
Desjardins, E. 2016. Private communication—cell marker value
Dhaun, N. et al., 2012. “Endothelin-1 and the kidney—beyond bp,” British Journal of Pharmacology, 167: 720-731.
DiPiro, J. T. et. al., 2010. “Concepts in clinical pharmacokinetics, 5^{th} ed.” McGraw-Hill, New York.
DiPiro, J. T. et. al., 2014. “Pharmacotherapy: a pathophysiological approach.” McGraw-Hill, New York.
EMD Millipore. 2016. “Pore size or NMWL (MWCO)”. Life Science Research.
Fauci et. al., 1998. Harrison's principles of internal medicine. McGraw-Hill, New York. p. 1508.
FiberCell Systems, Inc. 2014. “Endothelial Cartridge Instructions,” Revision 6.0, dated Jan. 8, 2014.
FiberCell Systems, Inc. 2015a. “Cartridge Product Insert.”.
FiberCell Systems. 2015b. “User Manual.”
Freshney, R. I. 2016. “Culture of Animal Cells, a Manual of Basic Technique and Specialized Applications”, 7th Edition, Wiley-Blackwell.
Ghayur, M., et al. 2008. “Contractility of the renal glomerulus and mesangial cells: lingering doubts and strategies for the future.” Med Hypotheses Res. 4 (1): 1-9.
G. E, Corp. 2015. “Cross-Flow Filtration Method Handbook.” p. 14
Guyton, A. C and J. E. Hall. 2013. “Textbook of medical physiology—11^{th} ed.,” Elsevier-Saunders, New York.
Kabgani et al., 2012. “Primary Cultures of Glomerular Parietal Epithelial Cells or Podocytes with Proven Origin,” PLoS ONE 7(4): 1-12, e34907.
Khan and Pandey, 2014. “Role of kidney biomarkers of chronic kidney disease: An update,” Saudi J. Biol. Sci., 21(4):294-299
Krtil et al., 2007. “Culture Methods of Glomerular Podocytes,” Kidney Blood Press Res., 30: 162-174.
Kumar, P. A., et al. 2014. “Molecular and cellular events mediating glomerular podocyte dysfunction and depletion in diabetes mellitus.” Frontiers in Endocrinology. 5: 1-10.
Langer, E. S. and R. A. Rader. 2014. Bioprocess Journal, Spring: 50-55.
Lee and Choi, 2012). “The artificial glomerulus design using diffusion in microchannels,” International Journal of Precision Engineering and Manufacturing, 13(2): 307-310.
Li et al. 2016. “Three-dimensional podocyte-endothelial cell co-cultures: Assembly, validation, and application to drug testing and intercellular signaling studies,” European Journal of Pharmaceutical Sciences, 86: 1-12.
Markovitz, M. “Artificial glomerulus and a method for treating blood.” U.S. Pat. No. 3,483,867, Jun. 13, 1968.
Miner, J. 2012. “The glomerular basement membrane,” Exp Cell Res, 318 (9): 973-978.
National Institute of Health. 2016a. “Glomerular filtration rate,” US National Library of Medicine.
National Institute of Health. 2016b. “Kidney tests,” US National Library of Medicine
Navarro-Gonzalez, J. and C. Morao-Fernandez. 2008. “The role of inflammatory cytokines in diabetic nephropathy,” J Am Soc Nephorl. 19: 433-422.
Neal, C. et al., 2007. “Glomerular filtration into the subpodocyte space is highly restricted under physiological perfusion conditions.” Am J Physiol Renal Physiol. 293: F1787-F1798.
Ni et al., 2012. “Podocyte culture: tricks of the trade,” Nephrology 17: 525-531.
Pall. 2016. “Choosing the correct MWCO.” Ultrafiltration fundamentals.
Pozzi, A. and R. Zent. 2013. “Integrins in kidney disease,” J Am Soc Nephrol, 24: 1034-1039.
Ristaldi, M. US Patent 0368608, Dec. 24, 2015.
Rastaldi, M and M. Li. “Method for the three-dimensional co-culture of podocytes and endothelial cells and relative in vitro co-culture system”. Patent Application Publication, Pub no.: US 2013.0177929 A1, Jul. 11, 2013.
Redmond, E.; Cahill, P.; et al., 1995. “Perfused transcapillary smooth muscle and endothelial cell co-culture—a novel in vitro model.” In Vitro Cell. Dev. Biol., 31: 601-9.
Redmond, E., Cahill, P. and James V. Sitzmann. 1998. “Flow-mediated regulation of G-protein expression in cocultured vascular smooth muscle and endothelial cells.” Arterioscler Thromb Vasc Biol., 18: 75-83.
Rops, A. 2004. “Isolation and characterization of conditionally immortalized mouse glomerular endothelial cell lines,” Kidney International, Vol. 66 (2004), pp. 2193-2201.
Sakairi et al., 2010. “Conditionally immortalized human podocyte cell lines established from urine,” Am J Physiol Renal Physiol., 298(3): F557-567.
Salmon A. H, et al., 2009. Curr Opin Nephrol Hypertens, 18(3): 197-205.
Sanyal, S. 2014. “Culture and assay systems used for 3D cell culture,” review article, Corning Life Sciences application note 245, document no. CLS-AC-AN-245 (publ. Corning Life Sciences) Sargent, B. 2013. “Perfusion bioreactors: with so much to offer they deserve a closer look.” Cell Culture Dish.
Satchell et al., 2006. “Conditionally immortalized human glomerular endothelial cells expressing fenestrations in response to VEGF,” Kidney International, 69: 1633-1640.
Schlondorff, D. 1996. “Roles of the mesangium in glomerular function,” Kidney International, 49: 1583-1585.
Schrijvers et al., 2004. “The role of vascular endothelial growth factor (VEGF) in renal pathophysiology,” Kidney International, 65: 2003-2017.
Scott, R. and S. Quaggin. 2016. “The cell biology of renal filtration,” J. Cell Biol., 209 (2): 199-210.
Sekulic, S. and M. Sekulic, 2013. “A compendium of urinary biomarkers indicative of glomerular Podocytopathy,” Pathology Research International, Volume 2013, Article ID 782395.
Sekulic, S. and M. Sekulic. 2015. “Rheological influence upon the glomerular podocyte and resultant mechanotransduction,” Kidney Blood Press Res, 40: 176-187.
Shankland et al., 2007. “Podocytes in culture: past, present, and future,” Kidney International 72(1): 26-36.
Singh, A. et al., 2007. “Glomerular endothelial glycocalyx constitutes a barrier to protein permeability,” J Am Soc Nephrol, 18: 2885-2893.
Slater et al., 2011. “An in vitro model of the glomerular capillary wall using electrospun collagen nanofibres in a bioartificial composite basement membrane,” PLoS ONE 6(6): e20802
Stockand, J. and S. Sansom. 1998. “Glomerular mesangial cells: electrophysiology and regulation of contraction.” Physiological Reviews, 78 (3): 723-744.
Sun et al. (2013), “Glomerular endothelial cell injury and damage precedes that of podocytes in adriamycin-induced nephropathy,” PLoS ONE 8(1): e55027.
Taneda et al. 2015, “Podocyte and endothelial injury in focal segmental glomerulosclerosis: an ultrastructural analysis,” Virchows Arch., 467(4): 449-458.
Tasnim et al., 2010. “Achievements and challenges in bioartificial kidney development,” Fibrogenesis & Tissue Repair 3:14
Thompson et al., (2015). “Complete and partial remission as surrogate end points in membranous nephropathy,” J Am Soc Nephrol. 26(12): 2930-2937
Truskey, G. A. et al. 2009. “Transport Phenomena in Biological Systems”. Pearson Education, New Jersey. Whitford, W. and Cadwell, J. “Interest in hollow-fiber perfusion bioreactors Is growing.” BioProcess Technical. October 2009, 54-63.
Zhang, C. et al., 2014. Journal of Diabetes Research, Article ID 953742.
Zinc and Li, “In vitro assay for predicting renal proximal tubule cell toxicity,” US Patent Appl. Publication No. US2015/0197802.
Zink and Zay. “Bioreactor unit for use in bioartificial kidney device,” US Patent Appl. Publication No. US2015/0076066.

While the foregoing disclosure has been described in some detail for purposes of clarity and understanding, it will be clear to one skilled in the art from a reading of this disclosure that various changes in form and detail can be made without departing from the true scope of the invention. It is to be understood that the Invention is not limited to any of the specifically recited methodologies, reagents, biological materials or instrumentation that are recited herein, where similar or equivalent methodologies, reagents, biological materials or instrumentation can be substituted and used in the construction and practice of the invention, and remain within the scope of the invention. It is understood that the description and terminology used in the present disclosure is for the purpose of describing particular embodiments of the invention only, and is not intended that the invention be limited solely to the embodiments described herein.

As used in this specification and the appended claims, singular forms such as “a”, “an” and “the” include plural referents unless the content clearly dictates otherwise. For example, reference to “a hollow fiber” also includes a plurality of hollow fibers; reference to “a cell” includes reference to a plurality of any number of cells, to cell cultures or to cell populations. All industry and technical terms used herein have the same meaning as commonly understood by one of ordinary skill in the art or industry to which the invention pertains, unless defined otherwise.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A partitioned cell co-culture comprising:
(a) an apparatus having:
i) an enclosed housing with an interior surface,
ii) at least one hollow fiber disposed within the enclosed housing, the at least one hollow fiber having
(A) a semi-permeable wall,
(B) an exterior surface and an interior surface, and
(C) an interior luminal space bounded by the interior surface of the hollow fiber, said luminal space being open at the ends of the at least one hollow fiber and comprising a directional liquid flow of a cell culture medium,
iii) at least one cell culture medium reservoir in fluid communication with the hollow fiber interior luminal space,
iv) an extra-capillary space bounded by the interior surface of the enclosed housing and the exterior surface of the hollow fiber, where the luminal space of the hollow fiber and the extra-capillary space are in diffusible communication, and
(b) glomerulus-derived vascular endothelial cells cultured within the luminal space and associated with the interior surface of the hollow fiber;
(c) podocyte cells cultured within the extra-capillary space and associated with the exterior surface of the hollow fiber.

2. The co-culture of claim 1, wherein the culture medium is selected from the group consisting of a whole blood, a defined cell culture medium, a defined cell culture medium supplemented with whole blood, a defined cell culture medium supplemented with a blood product, and a defined cell culture medium supplemented with a blood serum.

3. The co-culture of claim 1, wherein the cell culture medium is either (i) a mesangial cell-conditioned cell culture medium or mesangial cell-conditioned whole blood, or (ii) a cell culture medium that has been supplemented with a mesangial cell-conditioned cell culture medium or whole blood that has been supplemented with a mesangial cell-conditioned whole blood.

4. The co-culture of claim 1, further wherein the culture medium, i.e. whole blood comprises albumin protein, and wherein the semi-permeable wall of the hollow fiber retains at least 90% of albumin protein contained in the culture medium (health model).

5. The co-culture of claim 1, further wherein the culture medium, i.e. whole blood comprises albumin protein, and wherein the semi-permeable wall of the hollow fiber retains less than 90% of albumin protein contained in the culture medium (disease model).

6. The co-culture of claim 1, wherein the podocyte cells are selected from established podocyte cell lines and primary podocyte cell cultures.

7. The co-culture of claim 1, wherein the podocyte cells comprise at least one naturally-occurring genetic anomaly or at least one engineered genetic anomaly.

8. The co-culture of claim 1, wherein the podocyte cells are selected from porcine podocyte cells, marsupial podocyte cells, rodent podocyte cells, mouse podocyte cells, rat podocyte cells, mammalian podocyte cells, primate podocyte cells and human podocyte cells.

9. The co-culture of claim 1, wherein the glomerulus-derived vascular endothelial cells are selected from established glomerulus-derived vascular endothelial cell lines and primary glomerulus-derived vascular endothelial cell cultures.

10. The co-culture of claim 1, wherein the glomerulus-derived vascular endothelial cells comprise at least one naturally-occurring genetic anomaly or at least one engineered genetic anomaly.

11. The co-culture of claim 1, wherein the glomerulus-derived vascular endothelial cells are selected from porcine glomerulus-derived vascular endothelial cells, marsupial glomerulus-derived vascular endothelial cells, rodent glomerulus-derived vascular endothelial cells, mouse glomerulus-derived vascular endothelial cells, rat glomerulus-derived vascular endothelial cells, mammalian glomerulus-derived vascular endothelial cells, primate glomerulus-derived vascular endothelial cells and human glomerulus-derived vascular endothelial cells.

12. The co-culture of claim 1, wherein the exterior surface of the hollow fiber, the interior surface of the hollow fiber, or both, comprise an extracellular matrix coating selected from the group consisting of: natural (e.g. collagen IV α1α2α1, collagen IV α3α4α5)), naturally secreted or artificial (e.g. MAPTrix-C™ (AMSBIO)) proteins to coat the hollow fiber lumen surface, and/or selected from the group consisting of: natural (e.g. laminin-111, laminin-411), naturally secreted or artificial (e.g. MAPTrix-L™ (AMSBIO)) proteins coat the outer hollow fiber surface.

13. The co-culture of claim 1, wherein the apparatus further comprises a retentate reservoir that is in fluid communication with and downstream of the hollow fiber interior luminal space.

14. The co-culture of claim 1, wherein the apparatus further comprises a permeate reservoir that is in fluid communication with and downstream of the extra capillary space.

15. The co-culture of claim 1, wherein the apparatus further comprises a diafiltration reservoir that is in fluid communication with and upstream of the cell culture medium reservoir.

16. An apparatus for the co-culture of partitioned cell populations, the apparatus comprising:
(a) an enclosed housing with an interior surface,
(b) at least one hollow fiber disposed within the enclosed housing, the at least one hollow fiber having:
i) a semi-permeable wall having 90% retention efficiency for albumin protein contained in the culture medium (health model) or less than 90% retention efficiency for albumin protein (disease model),
ii) an exterior surface and an interior surface, and
iii) an interior luminal space bounded by the interior surface of the hollow fiber, said luminal space being open at the ends of the at least one hollow fiber,
(c) a culture medium reservoir in fluid communication with the hollow fiber interior luminal space,
(d) an extra-capillary space bounded by the interior surface of the enclosed housing and the exterior surface of the hollow fiber,
(e) a retentate reservoir that is in fluid communication with and downstream of the hollow fiber interior luminal space, and
(f) a permeate reservoir that is in fluid communication with and downstream of the extra capillary space.

17. The apparatus of claim 16, wherein the exterior surface of the hollow fiber, the interior surface of the hollow fiber, or both, comprise an extracellular matrix coating selected from the group consisting of: natural (e.g. collagen IV α1α2α1, collagen IV α3α4α5)), naturally secreted or artificial (e.g. MAPTrix-C™ (AMSBIO)) proteins to coat the hollow fiber lumen surface, and/or selected from the group consisting of: natural (e.g. laminin-111, laminin-411), naturally secreted or artificial (e.g. MAPTrix-L™ (AMSBIO)) proteins to coat the outer hollow fiber surface.

18. The apparatus of claim 16, further comprising a diafiltration reservoir that is in fluid communication with and upstream of the cell culture medium reservoir.

19. A method for assessing a cell physiological or system functional response to a treatment in a partitioned cell co-culture system, the method comprising the steps:
(A) culturing glomerulus-derived vascular endothelial cells and podocyte cells in a partitioned cell co-culture system, the system comprising:
i) an apparatus having:
(a) an enclosed housing with an interior surface,
(b) at least one hollow fiber disposed within the enclosed housing, the at least one hollow fiber having (I) a semi-permeable wall, (II) an exterior surface, (III) an interior surface, and (IV) an interior luminal space bounded by the interior surface of the hollow fiber, being open at the ends of the at least one hollow fiber,
(c) at least one culture medium reservoir comprising culture medium in fluid communication with the hollow fiber interior luminal space, and
(d) an extra-capillary space bounded by the interior surface of the enclosed housing and the exterior surface of the hollow fiber,
ii) a directional flow of the culture medium through the luminal space;
iii) glomerulus-derived vascular endothelial cells cultured within the luminal space and associated with the interior surface of the hollow fiber, and
iv) podocyte cells cultured within the extra-capillary space and associated with the exterior surface of the hollow fiber;
(B) assessing a cell physiological marker or a system functional measure prior to initiating a treatment in the glomerular cell co-culture model system;
(C) subjecting the glomerular cell co-culture model system (using whole blood) to a treatment includes one of: (i) altering a culture parameter or altering an equipment parameter to control or vary a biological engineering variable or (ii) delivery of a drug or drug candidate;
(D) subjecting the glomerular cell co-culture model system to a treatment further includes at least one control condition for each treatment;
(E) assessing the cell physiological marker or system functional measure at a time point(s) after initiating the treatment in the glomerular cell co-culture model system;
(F) comparing the cell physiological marker or system functional measure value prior to initiating the treatment to the cell physiological marker or system functional measure value at a time point(s) after initiating the treatment.
(G) comparing the cell physiological marker responses or the system functional measure responses to the treatment with the cell physiological responses or the system functional responses of the appropriate controls.

20. The method claim 19, wherein said subjecting the glomerular cell co-culture model system to treatment comprises equipment parameter alteration(s) selected from, but not limited to, one or more of a group consisting of: the feed flow rate, retentate control valve setting, tubing diameter and/or hollow fiber diameter, hollow fiber molecular weight cut off size. These are adjusted to control biological engineering variables including feed pressure, retentate pressure, average hollow fiber capillary pressure, shear rate, net filtration pressure and permeate flow rate to simulate various stages of glomerular disease.

21. The method claim 19, wherein said subjecting the glomerular cell co-culture model system to at least one control condition comprises equipment parameter alterations selected from, but not limited to, one or more of a group consisting of: the feed flow rate, retentate control valve setting, tubing diameter and/or hollow fiber diameter, hollow fiber molecular weight cut off size. These are adjusted to control biological engineering variables including feed pressure, retentate pressure, average hollow fiber capillary pressure, shear rate, net filtration pressure and permeate flow rate to simulate conditions of glomerular health.

22. The method claim 19, wherein said subjecting the glomerular cell co-culture model system to treatment comprises the delivery of a drug or drug candidate, in some embodiments pre-treated with e.g. liver tissue homogenate S9 subcellular fraction consists of:
(a) Determining the effects of particular drug(s) by delivery of i) whole blood and excipient with first drug; ii) whole blood and excipient with second drug; iii) whole blood with excipient and both first and second drug;
(b) Determining the effects of different doses of a particular drug by delivery of whole blood and excipient with different drug doses of a particular drug;
(c) Determining the effects of dosage intervals for a particular drug by delivery of whole blood and excipient with different dosage intervals for a particular drug and drug dose;
(a) Determining the effects of concentration gradients for a particular drug by delivery of whole blood and excipient with different drug concentration gradients (i.e. change in concentration over particular time interval).

23. The method claim 19, wherein said subjecting the glomerular cell co-culture model system to control condition(s) for the delivery of a drug or drug candidate, in some embodiments pre-treated with e.g. liver tissue homogenate S9 subcellular fraction comprises:
(b) Controlling for determining effects of particular drugs by delivery of: i) whole blood without excipient and without drug(s); ii) whole blood with excipient and without drug(s);
(c) Controlling for determining the effects of drug dosage by delivery of: i) whole blood without excipient and without drug; ii) whole blood with excipient and without drug;
(d) Controlling for determining the effects of drug dosage intervals by delivery of: i) whole blood without excipient and without drug; ii) whole blood with excipient and without drug for each dose interval to be tested; iv) constant flat line delivery of drug dose in whole blood with excipient;
(e) Controls for determining the effects of concentration gradients (i.e. change in concentration over a time interval) of a drug by delivery of: i) whole blood without excipient and without drug; ii) whole blood with excipient and without drug for each gradient to be tested; iii) constant flat line delivery of excipient and representative concentrations of drug used in the particular gradient tested for the time interval of that gradient for each concentration gradient tested.

24. The method of claim 19, wherein the exterior surface of the hollow fiber, the interior surface of the hollow fiber, or both surfaces of the hollow fiber comprise an extracellular matrix coating selected from the group consisting of: natural (e.g. collagen IV α1α2α1, collagen IV α3α4α5)), naturally secreted or artificial (e.g. MAPTrix-C™ (AMSBIO)) proteins to coat the hollow fiber lumen surface, and/or selected from the group consisting of: natural (e.g. laminin-111, laminin-411), naturally secreted or artificial (e.g. MAPTrix-L™ (AMSBIO)) proteins to coat the outer hollow fiber surface.

25. The co-culture of claim 19, wherein the apparatus further comprises a retentate reservoir that is in fluid communication with and downstream of the hollow fiber interior luminal space.

26. The co-culture of claim 19, wherein the apparatus further comprises a permeate reservoir that is in fluid communication with and downstream of the extra capillary space.

27. The method of claim 19, wherein the apparatus further comprises a diafiltration reservoir that is in fluid communication with and upstream of the culture medium reservoir.

28. The method of claim 19, wherein the apparatus further comprises a first drug reservoir that is in fluid communication with and upstream of the culture medium reservoir.

29. The method of claim 19, wherein the apparatus further comprises a second drug reservoir that is in fluid communication with and upstream of the culture medium reservoir.

30. The method of claim 19, wherein the apparatus further comprises a diluent (medium without drug, i.e. whole blood) reservoir that is in fluid communication with and upstream of the culture medium reservoir.

31. The method claim 19, wherein said system functional measure is the total protein sieving coefficient or permeate total protein concentration.

32. The method claim 19, wherein said system functional measure is an albumin sieving coefficient or albumin permeate concentration.

33. The method claim 19, wherein said system functional measure is the flow rate of a liquid fraction passing through the semi-permeable wall of the hollow fiber into the extra-capillary space.

34. The method claim 19, wherein said system functional measure is the red blood cell count/mL of permeate.

35. The method claim 19, wherein said cell physiological marker is a glomerulus-derived vascular endothelial cell marker.

36. The method claim 19, wherein said cell physiological marker is a podocyte cell marker.

37. The method claim 19, wherein said cell physiological marker is a circulating molecular marker in the cell culture medium.

38. The method claim 19, wherein said cell physiological marker is selected from a polypeptide marker and a nucleic acid marker
